(19) United States
(12) Patent Application Publication
(10) Pub. No.: US 2014/0087474 A1
(43) Pub. Date: Mar. 27, 2014

(54) APPARATUS AND METHODS FOR PERFORMING OPTICAL NANOPORE DETECTION OR SEQUENCING

(71) Applicant: Quantapore, Inc., Menlo Park, CA (US)

(72) Inventor: Martin HUBER, Menlo Park, CA (US)

(73) Assignee: Quantapore, Inc., Menlo Park, CA (US)

(21) Appl. No.: 14/018,376

(22) Filed: Sep. 4, 2013

Related U.S. Application Data
(63) Continuation-in-part of application No. PCT/US2011/054365, filed on Sep. 30, 2011.
(60) Provisional application No. 61/449,531, filed on Mar. 4, 2011.

(51) Int. Cl. C12Q 1/68

(52) U.S. Cl. CPC C12Q 1/6869
USPC 436/94

(57) ABSTRACT

Methods and systems for detecting or sequencing a biological molecule or polymer, e.g., a nucleic acid, are provided. Optical sequencing of a molecule may be performed utilizing an optical or photon detector operated in time delayed integration mode. In certain variations, the translocation rate of molecules through a, pore, nanopore or channel may be controlled or reduced by increasing the diameter of the molecules to allow for molecule detection or sequencing by optical or electrical detection. In certain variations, a plurality or an array of, pores, nanopores, or channels may be utilized to optically detect a plurality of molecules.

### CROSS-REFERENCE TO RELATED APPLICATION

This application is a continuation-in-part of PCT application number PCT/US2011/054365 filed Sep. 30, 2011, which claims the benefit of priority to U.S. Prov. Pat. App. 61/449,531 filed Mar. 4, 2011, the contents of each of which are incorporated by reference herein in their entireties. U.S. application Ser. No. 13/426,515, filed Mar. 21, 2012, U.S. Application No. 61/277,939, filed Sep. 30, 2009, and PCT Application No. PCT/US2010/034809, filed May 13, 2010 are also incorporated herein by reference in their entireties.

### BACKGROUND

DNA is a long bio-polymer made from repeating units called nucleotides. DNA polymers can be enormous molecules containing millions of nucleotides e.g. the human genome contains a total of 3 billion nucleotides. In living organisms, DNA does not usually exist as a single molecule, but instead as a tightly-associated pair of molecules. These two long strands intertwine like vines in the shape of a double helix. The nucleotide repeats contain both a phosphate backbone which holds the chain together, and a base which interacts with the other DNA strand in the helix. This interaction between the bases of the two DNA strands is called hydrogen bonds and they hold the double helix together. There are four different types of bases: Adenine (A), Cytosine (C), Guanine (G) and Thymine (T). Each type of base in one strand forms a hydrogen bond with just one type of base in the complementary strand, with A bonding only to T, and C bonding only to G.

The sequence of the four bases determines the genetic information contained in DNA. Revealing the sequence of the four building blocks of polynucleic acid is called sequencing. Polynucleic acid comprises bases of nucleosides chemically bound in a linear fashion. “DNA” (De-oxyribonucleic acid) and “RNA” (Ribonucleic acid) are examples of such polynucleic acid molecules. The particular order or “sequence” of these bases in a given gene determines the structure of the protein encoded by the gene. Furthermore, the sequence of bases surrounding the gene typically contains information about how often the particular protein should be made, in which cell types etc.

The complete nucleotide sequence of all DNA polymers in a particular individual is known as that individual's “genome”. In 2003 the human genome project was finished and a draft version of the human DNA sequence was presented. It took 13 years, US$3 billion and the joint power of multiple sequencing centers to achieve this scientific milestone which was compared in significance to the arrival of men on the moon. The method used for this giant project is called Sanger sequencing (Sanger, F. et al., Proc. Natl. Acad. Sci. USA (1977) 74, 5463-5467 and Smith et al., U.S. Pat. No. 5,821,058). Although major technical improvements were made during this time, the classical sequencing method has some key-disadvantages, namely, laborious sample preparation, including subcloning of DNA fragments in bacteria, expensive automation, cost prohibitive molecular biology reagents, and limited throughput which results in weeks to finish sequencing whole genomes.

Multiple diseases have a strong genetic component (Strittmatter, W. J. et al., Annual Review of Neuroscience 19 (1996): 53-77; Ogura, Y. et al., Nature 411, (2001): 603-606; Begovich, A. B. et al., American Journal of Human Genetics 75, (2004): 330-337). With the completion of the Human Genome Project and an ever deepening comprehension of the molecular basis of disease, medicine in the 21st century is poised for a revolution called “molecular diagnostics”. Most commercial and academic approaches in molecular diagnostics assess single nucleotide variations (SNPs) or mutations to identify DNA aberrations. These technologies, although powerful, will analyze only a small portion of the entire genome. The inability to accurately and rapidly sequence large quantities of DNA remains an important bottleneck for research and drug development (Shaffer, C., Nat Biotech 25 (2007): 149).

Genomic technologies are emerging as the most revolutionary scientific breakthrough of this century. Because a wealth of knowledge contained in the genome of an organism can be unlocked by knowing the sequence of the DNA, fast and cheap ways of performing DNA sequencing are extremely crucial for the development of this field. Nanopore based methods allow the simplest and most elegant method for DNA sequencing. Briefly, the DNA is passed through a nanopore whose width is of the order of the width of DNA. The advantages of nanopore based methods lie in the fact that no enzymes are required to read out the DNA sequence, the high detection speed, the lack of sample preparation prior to the sequencing reaction which combined result in a high-throughput and low cost sequencing technology. Electrical detection schemes have been most commonly used for nanopore sequencing applications, they are, however, severely limited by the noise-floor of the high-bandwidth amplifiers, the finite width of any embedded nanoelectrodes, the difficulty or inability to parallelize asynchronous high-speed detection events or to differentiate or detect nucleic acids translocated at high sampling rates, and the inability to control the translocation speed of nucleic acids. Clearly, there is a need for the development of improved sequencing technologies that are faster, easier to use, more accurate and less expensive.

### BRIEF SUMMARY

Variations described herein relate to methods, systems and/or devices for detecting various molecules, e.g., methods and systems for optical detection or sequencing of molecules or polymers or for detection or sequencing of a composition of biological molecules or polymers. For example, methods and devices are described herein which are capable of ultrafast polymer detection or sequencing utilizing a labeled pore or nanopore and a biological polymer with labeled monomer building blocks.

Methods and systems for sequencing a biological molecule or polymer, e.g., a nucleic acid, are provided. One or more donor labels, which are positioned on, attached or connected to a pore or nanopore, may be illuminated or otherwise excited. A polymer labeled with one or more acceptor labels, may be translocated through the nanopore. For example, a polymer having one or more monomers labeled with one or more acceptor labels, may be translocated through the nanopore. Either before, after or while the labeled monomer of the polymer or molecule passes through, exits or enters the nanopore and when an acceptor label comes into proximity with a donor label, energy may be transferred from the excited donor label to the acceptor label of the monomer or polymer. As a result of the energy transfer, the acceptor label emits energy, and the emitted energy may be detected or measured, e.g., using an optical detector, in order to identify the specific monomer, e.g., the nucleotides of a translocated nucleic acid molecule, which is associated with the detected acceptor label energy or photon emission. The nucleic acid or other polymer may be deduced or sequenced based on the detected or measured energy or photon emission from the acceptor labels and the identification of the monomers or monomer sub units associated therewith.

Various methods and systems for detecting or sequencing a biological molecule or polymer, e.g., a nucleic acid, are described herein. In certain variations, detecting or sequencing of a molecule may be performed utilizing one or more optical or photon detectors. The photon detector may optionally be operated in time delayed integration mode. In certain variations, the translocation rate of one or more molecules through a nanopore, pore or channel may be controlled or reduced by increasing the diameter of the molecule to allow for molecule detection or sequencing by optical and/or electrical detection. In certain variations, a plurality or an array of nanopores may be utilized to optically detect a plurality of molecules simultaneously.

In certain variations, a method of optically detecting a molecule may include one or more of the following steps. One or more nanopores, pores or channels may be provided on a substrate. A labeled molecule may be translocated through the nanopore, pore or channel. An energy emission from the labeled molecule is detected using an optical detector, e.g., operated in time delay integration mode or another mode, where the energy emission is associated with a specific molecule. The identity of the molecule may be deduced based on detection of the energy emission.

In certain variations, a method of optically detecting a plurality of molecules may include one or more of the following steps. A plurality or array of nanopores or channels may be provided. A plurality of molecules may be translocated through the nanopores, pores or channels. A separate energy emission may be optically detected from each molecule at each nanopore, pore or channel simultaneously, wherein each energy emission has a wavelength associated with a specific molecule. The molecules may be identified based on the detected energy emissions.

In certain variations, a system for optically detecting a plurality of molecules may include a plurality or array of nanopores, pores or channels arranged on a substrate and one or more optical detectors. The optical detectors may be configured to detect a separate energy emission from each molecule at each nanopore, pore or channel simultaneously, wherein each energy emission has a wavelength associated with a specific molecule.

In certain variations, a method of reducing the translocation speed of a molecule through a nanopore, pore or channel to allow for detection of the molecule may include one or more of the following steps. The diameter of the molecule may be increased by modifying the molecule. A single molecule may be translocated through the nanopore, pore or channel where the increased diameter of the molecule causes the molecule to have a reduced translocation speed, e.g., by interacting with the nanopore, pore or channel, to reduce the speed at which the molecule translocates through the nanopore, pore or channel.

In certain variations, a method of optically sequencing a nucleic acid may include providing a substrate having a plurality of nanopores. A plurality of labeled nucleic acids may be translocated in single file through the plurality of nanopores simultaneously. Separate energy emissions may be detected from each labeled nucleotide of a nucleic acid simultaneously after the labeled nucleotide passes through and exits the nanopore using an optical detector. Optionally, the optical detector may be operated in time delay integration mode or another mode. An energy emission may be associated with a specific nucleotide and the optical detector may differentiate between each detected energy emission and detect at which nanopore the energy is emitted. The nucleic acid may be sequenced based on detection of the energy emissions from the labeled nucleotides. In certain variations, the energy emissions may be processed or converted to a signal and read, e.g., utilizing various software or detection systems, to sequence the nucleic acid.

### BRIEF DESCRIPTION OF THE SEVERAL VIEWS OF THE DRAWINGS

FIG. 1A illustrates a variation of a synthetic nanopore having a pore label attached thereto.

FIG. 1B illustrates a variation of a protein nanopore having a pore label attached thereto.

FIG. 2A illustrates one variation of a FRET (Förster Resonance Energy Transfer) interaction between a pore label on a synthetic nanopore and a nucleic acid label on a nucleic acid which is being translocated through the synthetic nanopore.

FIG. 2B illustrates translocation of the labeled nucleic acid through a synthetic nanopore at a point in time where no FRET is taking place.

FIG. 2C illustrates one variation of a FRET interaction between a pore label on a protein nanopore and a nucleic acid label on a nucleic acid which is being translocated through the protein nanopore.

FIG. 2D illustrates translocation of a labeled nucleic acid through a protein nanopore at a point in time where no FRET is taking place.

FIG. 3 illustrates one variation of a multicolor FRET interaction between the donor labels (Quantum dots) of a protein nanopore and the acceptor labels of a nucleic acid. Each shape on the nucleic acid represents a specific acceptor label, where each label has a distinct emission spectra associated with a specific nucleotide such that each label emits light at a specific wavelength associated with a specific nucleotide.

FIG. 4A illustrates partial contigs from nucleic acid sequencing utilizing a singly labeled nucleic acid.

FIG. 4B illustrates how partial contig alignment may generate a first draft nucleic acid sequence.

FIG. 5A illustrates one variation of a quenching interaction between a pore label on a synthetic nanopore and a nucleic acid label on a nucleic acid which is being translocated through the synthetic nanopore.

FIG. 5B illustrates translocation of the labeled nucleic acid through a synthetic nanopore at a point in time where no quenching is taking place.

FIG. 5C illustrates one variation of a quenching interaction between a pore label on a protein nanopore and a nucleic acid label on a nucleic acid which is being translocated through the protein nanopore.

FIG. 5D illustrates translocation of a labeled nucleic acid through a protein nanopore at a point in time where no quenching is taking place.

FIG. 6 shows an example of an absorption/emission spectra from a FRET pair containing a donor quantum dot and an acceptor fluorophore.

FIGS. 7A and 7B show a schematic of a variation of a system for performing optical pore or nanopore molecule detection or sequencing,

FIGS. 7C and 7D show a schematic of the detection of temporal information from a linear array of nanopores.

FIG. 8 shows a schematic of another variation of a system for performing optical nanopore molecule detection or sequencing.

FIGS. 9A-9C show variations of substrates including arrays of pores or nanopores in various configurations.

### DETAILED DESCRIPTION

A method and/or system for sequencing a biological polymer or molecule (e.g., a nucleic acid) may include exciting one or more donor labels attached to a pore or nanopore. A biological polymer may be translocated through the pore or nanopore, where a monomer of the biological polymer is labeled with one or more acceptor labels. Energy may be transferred from the excited donor label to the acceptor label of the monomer as, after or before the labeled monomer passes through, exits or enters the pore or nanopore. Energy emitted by the acceptor label as a result of the energy transfer may be detected, where the energy emitted by the acceptor label may correspond to or be associated with a single or particular monomer (e.g., a nucleotide) of a biological polymer. The sequence of the biological polymer may then be deduced or sequenced based on the detection of the emitted energy from the monomer acceptor label which allows for the identification of the labeled monomer. A pore, nanopore, channel or passage, e.g., an ion permeable pore, nanopore, channel or passage may be utilized in the systems and methods described herein.

Nanopore energy transfer sequencing (NETS) can be used to sequence nucleic acid. NETS can enable the sequencing of whole genomes within days for a fraction of today's cost which will revolutionize the understanding, diagnosis, monitoring and treatment of disease. The system or method can utilize a pore or nanopore (synthetic or protein-based) of which one side, either the cis (−) or trans (+) side of the pore is labeled with one or multiple or a combination of different energy absorbers or donor labels, such as fluorophores, fluorescent proteins, quantum dots, metal nanoparticles, nanodiamonds, etc. Multiple labels and methods of labeling a nanopore are described in U.S. Pat. No. 6,528,258, the entirety of which is incorporated herein by reference.

A nucleic acid can be threaded through a nanopore by applying an electric field through the nanopore (Kasianowicz, J. J. et al., Characterization of individual polynucleotide molecules using a membrane channel. Proc. Natl. Acad. Sci. USA 93 (1996): 13770-13773). A nucleic acid to be translocated through the nanopore may undergo a labeling reaction where naturally occurring nucleotides are exchanged with a labeled, energy emitting or absorbing counterpart or modified counterparts that can be subsequently modified with an energy emitting or absorbing label., i.e., an acceptor label. The labeled nucleic acid may then be translocated through the nanopore and upon entering, exiting or while passing through the nanopore a labeled nucleotide comes in close proximity to the nanopore or donor label. For example, within 1-10 nm or 1-2 nm of the nanopore donor label. The donor labels may be continuously illuminated with radiation of appropriate wavelength to excite the donor labels. Via a dipole-dipole energy exchange mechanism called FRET (Stryer, L. Annu Rev Biochem. 47 (1978): 819-846), the excited donor labels transfer energy to a bypassing nucleic acid or acceptor label. The excited acceptor label may then emit radiation, e.g., at a lower energy than the radiation that was used to excite the donor label. This energy transfer mechanism allows the excitation radiation to be “focused” to interact with the acceptor labels with sufficient resolution to generate a signal at the single nucleotide scale.

A nanopore or pore may include any opening or channel positioned in a substrate that allows the passage of a molecule through the substrate. For example, the nanopore may allow passage of a molecule that would otherwise not be able to pass through that substrate. Examples of nanopores include proteinaceous or protein based pores or synthetic pores. A nanopore or pore may have an inner diameter of 1-10 nm or 1-5 nm or 1-3 nm.

Examples of protein pores include but are not limited to, alpha-hemolysin, voltage-dependent mitochondrial porin (VDAC), OmpF, OmpC, MspA and LamB (maltoporin) (Rhee, M. et al., Trends in Biotechnology, 25(4) (2007): 174-181). Any protein pore that allows the translocation of single nucleic acid molecules may be employed. A pore protein may be labeled at a specific site on the exterior of the pore, or at a specific site on the exterior of one or more monomer units making up the pore forming protein.

A synthetic pore may be created in various forms of solid substrates, examples of which include but are not limited to silicones (e.g. Si3N4, SiO2), metals, metal oxides (e.g. Al2O3) plastics, glass, semiconductor material, graphene, and combinations thereof. A synthetic nanopore may be more stable than a biological protein pore positioned in a lipid bilayer membrane.

Synthetic nanopores may be created using a variety of methods. For example, synthetic nanopores may be created by ion beam sculpting (Li, J. et al., Nature 412 (2001): 166-169) where massive ions with energies of several thousand electron volts (eV) cause an erosion process when fired at a surface which eventually will lead to the formation of a nanopore. A synthetic nanopore may be created via latent track etching. For example, a single conical synthetic nanopore may be created in a polymer substrate by chemically etching the latent track of a single, energetic heavy ion. Each ion produces an etchable track in a polymer foil, forming a one-pore membrane (Heins. E. A. et al., Nano Letters 5 (2005): 1824-1829). A synthetic nanopore may also be created by a method called Electron beam-induced fine tuning. Nanopores in various materials have been fabricated by advanced nanofabrication techniques, such as FIB drilling and electron (E) beam lithography, followed by E-beam assisted fine tuning techniques. With the appropriate electron beam intensity applied, a previously prepared nanopore will start to shrink. The change in pore diameter may be monitored in real-time using a TEM (transmission electron microscope), providing a feedback mechanism to switch off the electron beam at any desired dimension of the nanopore (Lo, C. J. et al., Nanotechnology 17 (2006): 3264-67).

A synthetic nanopore may also be created by using a carbon nanotube embedded in a suitable substrate such as but not limited to polymerized epoxy. Carbon nanotubes may have uniform and well-defined chemical and structural properties. Various sized carbon nanotubes can be obtained, ranging from one to hundreds of nanometers. The surface charge of a carbon nanotube is known to be about zero, and as a result, electrophoretic transport of a nucleic acid through the nanopore becomes simple and predictable (Ito, T. et al., Chem. Commun. 12 (2003): 1482-83).

A pore may have two sides. One side is referred to as the “cis” side and faces the (−) negative electrode or a negatively charged buffer/ion compartment or solution. The other side is referred to as the “trans” side and faces the (+) electrode or a positively charged buffer/ion compartment or solution. A biological polymer, such as a labeled nucleic acid molecule or polymer can be pulled or driven through the pore by an electric field applied through the nanopore, e.g., negatively charged DNA entering on the cis side of the nanopore and exiting on the trans side of the nanopore. For a positively charged polymer the translocation direction is reversed.

A nanopore or pore may be labeled with one or more donor labels. For example, the cis side or surface and/or trans side or surface of the nanopore may be labeled with one or more donor labels. The label may be attached to the base of a pore or nanopore or to another portion or monomer making up the nanopore or pore. A label may be attached to a portion of the membrane or substrate through which a nanopore spans or to a linker or other molecule attached to the membrane, substrate or nanopore. The nanopore or pore label may be positioned or attached on the nanopore, substrate or membrane such that the pore label can come into proximity with an acceptor label of a biological polymer, e.g., a nucleic acid, which is translocated through the pore. The donor labels may have the same or different emission or absorption spectra.

The labeling of a pore structure may be achieved via covalent or non-covalent interactions. Examples of such non-covalent interactions include but are not limited to interactions based on hydrogen bonds, hydrophobic interactions, electrostatic interactions, ionic interactions, magnetic interactions, Van der Walls forces or combinations thereof.

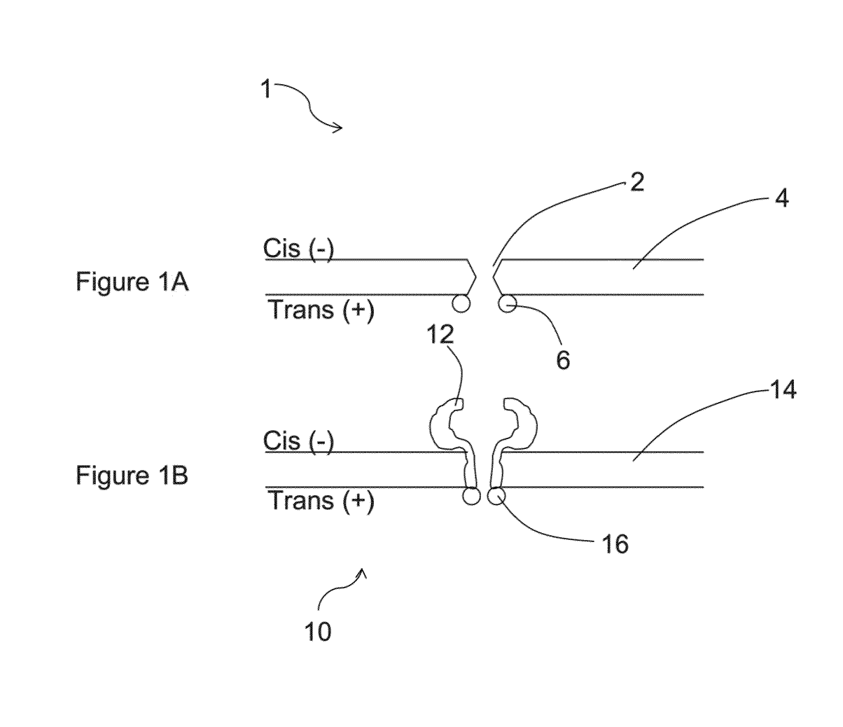

A donor label may be placed as close as possible to the aperture of a nanopore without causing an occlusion that impairs translocation of a nucleic acid through the nanopore (see e.g., FIG. 1). A pore label may have energy absorption properties meeting particular requirements. A pore label may have a large radiation energy absorption cross-section, ranging, for example, from about 0 to 1000 nm or from about 200 to 500 nm. A pore label may absorb radiation within a specific energy range that is higher than the energy absorption of the nucleic acid label. The absorption energy of the pore label may be tuned with respect to the absorption energy of a nucleic acid label in order to control the distance at which energy transfer may occur between the two labels. A pore label may be stable and functional for at least 10̂6 or 10̂9 excitation and energy transfer cycles.

FIG. 1A shows a variation of a pore/substrate assembly 1. The pore/substrate assembly 1 includes a synthetic pore or nanopore 2 which has a pore label 6 attached thereto. The assembly may also include a substrate 4, e.g., a solid substrate, and the synthetic nanopore 2 is positioned in the substrate 4. The synthetic nanopore 2 is modified at the trans (+) side with one or more pore labels 6. The pore label 6 is attached to the base of the synthetic nanopore 2 in a manner such that the label 6 does not lead to inclusion or impair the translocation of a nucleic acid through the synthetic nanopore 2.

FIG. 1B shows a variation of a pore/lipid bilayer assembly 10. The pore/lipid bilayer assembly 10 includes a protein nanopore 12 which has a pore label 16 attached thereto. The assembly may also include a lipid bilayer 14 and the protein nanopore 12 is positioned in the lipid bilayer 14. The protein nanopore 12 is modified at the trans (+) side with one or more pore labels 16. The pore label 16 is attached to the base of the protein nanopore 12 in a manner such that the label 16 does not lead to inclusion or impair the translocation of a nucleic acid through the protein nanopore 12.

A protein nanopore can be embedded in a phospholipid bilayer or derivatizations thereof. Phospholipids are comprised of, but not limited to diphytanoyl-phospatidylcoline, soybean azolectin, 1,2-Diphytanoyl-sn-glycero-3-phosphocholine. A lipid bilayer can also be prepared by a mixture of different phospholipids. Possible solvents for phospholipids are hexadecane, pentane, chloroform or any other suitable organic solvent. A lipid bilayer may be prepared in variety of ways known to those having ordinary skill in the art.

A lipid bilayer (e.g., including the above mentioned phospholipids) having a pore protein may be prepared according to the following method: A 10-25 μm thick Teflonfilm (Septum) with a 1-100 μm aperture separates two buffer compartments made out of Teflon. The septum and/or aperture can be primed with 10% hexadecane in pentane on each side and after evaporation of the solvent the buffer compartments are filled with 1 molar KCl. 1,2-Diphytanoyl-sn-glycero-3-phosphocholine (Avanti, 10 mg/mL in pentane) can be added to each buffer compartment and the pentane is allowed to evaporate leaving behind lipid monolayers. The liquid level in the chamber can be raised and lowered below and above the aperture, and can cause lipid bilayers to be formed. The formation of the bilayer can be measured by applying voltage via Ag/AgCl electrodes.

Once the bilayer has formed the ionic current is completely eliminated. With the bilayer in place a dilute solution of pore protein is added to the cis-chamber. Pore proteins can be chosen from a group of proteins such as, but not limited to, alpha-hemolysin, voltage-dependent mitochondrial porin (VDAC), Anthrax porin, OmpF, OmpC and LamB (maltoporin). The pore can self-assemble and integrate into the lipid bilayer. Integration of the pore protein can be measured by a small but constant current. One inserted hemolysin pore can carry an ionic current of approximately 120 pA (picoAmperes), with an applied voltage of +120 mV (milliVolts). The membrane can be protected by a second layer of a polymeric structure, comprising but not limited to Agarose, Polyacrylamide, etc. (Kang, X.-F. et al., J Am Chem Soc. 129 (2007): 4701-4705).

Various polymers or molecules may be attached to a lipid bilayer having a pore protein therein to provide additional stability and support to the pore/membrane assembly and the pore should the membrane be damaged.

A pore label may include one or more Quantum dots. A Quantum dot has been demonstrated to have many or all of the above described properties and characteristics found in suitable pore labels (Bawendi M. G. in U.S. Pat. No. 6,251,303). Quantum Dots are nanometer scale semiconductor crystals that exhibit strong quantum confinement due to the crystals radius being smaller than the Bohr exciton radius. Due to the effects of quantum confinement, the bandgap of the quantum dots increases with decreasing crystal size thus allowing the optical properties to be tuned by controlling the crystal size (Bawendi M. G. et al., in U.S. Pat. No. 7,235,361 and Bawendi M. G. et al., in U.S. Pat. No. 6,855,551).

One example of a Quantum dot which may be utilized as a pore label is a CdTe quantum dot which can be synthesized aqueously. A CdTe quantum dot may be functionalized with a nucleophilic group such as primary amines, thiols or functional groups such as carboxylic acids. A CdTe quantum dot may include a mercaptopropionic acid capping ligand, which has a carboxylic acid functional group that may be utilized to covalently link a quantum dot to a primary amine on the exterior of a protein pore. The cross-linking reaction may be accomplished using standard cross-linking reagents (homo-bifunctional as well as hetero-bifunctional) which are known to those having ordinary skill in the art of bioconjugation. Varying the length of the employed crosslinker molecule used to attach the donor label to the nanopore can, for example, ensure that the modifications do not impair or substantially impair the translocation of a nucleic acid through the nanopore.

The primary amine of the Lysin residue 131 of the natural alpha hemolysin protein (Song, L. et al., Science 274, (1996): 1859-1866) may be used to covalently bind carboxy modified CdTe Quantum dots via 1-Ethyl-3-[3-dimethylaminopropyl]carbodiimide hydrochloride/N-hydroxysulfosuccinimide (EDC/NHS) coupling chemistry.

A variety of methods, mechanisms and/or routes for attaching one or more pore labels to a pore protein may be utilized. A pore protein may be genetically engineered in a manner that introduces amino acids with known properties or various functional groups to the natural protein sequence. Such a modification of a naturally occurring protein sequence may be advantageous for the bioconjugation of Quantum dots to the pore protein. For example, the introduction of a Cystein residue would introduce a thiol group that would allow for the direct binding of a Quantum dot, such as a CdTe quantum dot, to a pore protein. Also, the introduction of a Lysin residue would introduce a primary amine for binding a Quantum dot. The introduction of glutamic acid or aspartic acid would introduce a carboxylic acid moiety for binding a Quantum dot. These groups are amenable for bioconjugation with a Quantum dot using either homo- or hetero-bifunctional crosslinker molecules. Such modifications to pore proteins aimed at the introduction of functional groups for bioconjugation are known to those having ordinary skill in the art. Care should be taken to ensure that the modifications do not impair or substantially impair the translocation of a nucleic acid through the nanopore.

The nanopore label can be attached to a protein nanopore before or after insertion of said nanopore into a lipid bilayer. Where a label is attached before insertion into a lipid bilayer, care may be taken to label the base of the nanopore and avoid random labeling of the pore protein. This can be achieved by genetic engineering of the pore protein to allow site specific attachment of the pore label (see section 0047). An advantage of this approach is the bulk production of labeled nanopores. Alternatively, a labeling reaction of a pre-inserted nanopore may ensure site-specific attachment of the label to the base (trans-side) of the nanopore without genetically engineering the pore protein.

A biological polymer, e.g., a nucleic acid molecule or polymer, may be labeled with one or more acceptor labels. For a nucleic acid molecule, each of the four nucleotides or building blocks of a nucleic acid molecule may be labeled with an acceptor label thereby creating a labeled (e.g., fluorescent) counterpart to each naturally occurring nucleotide. The acceptor label may be in the form of an energy accepting molecule which can be attached to one or more nucleotides on a portion or on the entire strand of a converted nucleic acid.

A variety of methods may be utilized to label the monomers or nucleotides of a nucleic acid molecule or polymer. A labeled nucleotide may be incorporated into a nucleic acid during synthesis of a new nucleic acid using the original sample as a template (“labeling by synthesis”). For example, the labeling of nucleic acid may be achieved via PCR, whole genome amplification, rolling circle amplification, primer extension or the like or via various combinations and extensions of the above methods known to persons having ordinary skill in the art.

Labeling of a nucleic acid may be achieved by replicating the nucleic acid in the presence of a modified nucleotide analog having a label, which leads to the incorporation of that label into the newly generated nucleic acid. The labeling process can also be achieved by incorporating a nucleotide analog with a functional group that can be used to covalently attach an energy accepting moiety in a secondary labeling step. Such replication can be accomplished by whole genome amplification (Zhang, L. et al., Proc. Natl. Acad. Sci. USA 89 (1992): 5847) or strand displacement amplification such as rolling circle amplification, nick translation, transcription, reverse transcription, primer extension and polymerase chain reaction (PCR), degenerate oligonucleotide primer PCR (DOP-PCR) (Telenius, H. et al., Genomics 13 (1992): 718-725) or combinations of the above methods.

A label may comprise a reactive group such as a nucleophile (amines, thiols etc.). Such nucleophiles, which are not present in natural nucleic acids, can then be used to attach fluorescent labels via amine or thiol reactive chemistry such as NHS esters, maleimides, epoxy rings, isocyanates etc. Such nucleophile reactive fluorescent dyes (i.e. NHS-dyes) are readily commercially available from different sources. An advantage of labeling a nucleic acid with small nucleophiles lies in the high efficiency of incorporation of such labeled nucleotides when a “labeling by synthesis” approach is used. Bulky fluorescently labeled nucleic acid building blocks may be poorly incorporated by polymerases due to sterical hindrance of the labels during the polymerization process into newly synthesized DNA.

DNA can be directly chemically modified without polymerase mediated incorporation of labeled nucleotides. One example of a modification includes cis-platinum containing dyes that modify Guanine bases at their N7 position (Hoevel, T. et al., Bio Techniques 27 (1999): 1064-1067). Another example includes the modifying of pyrimidines with hydroxylamine at the C6 position which leads to 6-hydroxylamino derivatives. The resulting amine groups can be further modified with amine reactive dyes (e.g. NHS-Cy5).

A nucleic acid molecule may be directly modified with N-Bromosuccinimide which upon reacting with the nucleic acid will result in 5-Bromocystein, 8-Bromoadenine and 8-Bromoguanine. The modified nucleotides can be further reacted with di-amine nucleophiles. The remaining nucleophile can then be reacted with an amine reactive dye (e.g. NHS-dye) (Hermanson G. in BiocojugateTechniques, Academic Press 1996, ISBN 978-O-12-342336-8).

A combination of 1, 2, 3 or 4 nucleotides in a nucleic acid strand may be exchanged with their labeled counterpart. The various combinations of labeled nucleotides can be sequenced in parallel, e.g., labeling a source nucleic acid or DNA with combinations of 2 labeled nucleotides in addition to the four single labeled samples, which will result in a total of 10 differently labeled sample nucleic acid molecules or DNAs (G, A, T, C, GA, GT, GC, AT, AC, TC). The resulting sequence pattern may allow for a more accurate sequence alignment due to overlapping nucleotide positions in the redundant sequence read-out.

A method for sequencing a polymer, such as a nucleic acid molecule includes providing a nanopore or pore protein (or a synthetic pore) inserted in a membrane or membrane-like structure or other substrate. The base or other portion of the pore may be modified with one or more pore labels. The base may refer to the Trans side of the pore. Optionally, the Cis and/or Trans side of the pore may be modified with one or more pore labels. Nucleic acid polymers to be analyzed or sequenced may be used as a template for producing a labeled version of the nucleic acid polymer, in which one of the four nucleotides or up to all four nucleotides in the resulting polymer is/are replaced with the nucleotide's labeled analogue(s). An electric field is applied to the nanopore which forces the labeled nucleic acid polymer through the nanopore, while an external monochromatic or other light source may be used to illuminate the nanopore, thereby exciting the pore label. As, after or before labeled nucleotides of the nucleic acid pass through, exit or enter the nanopore, energy is transferred from the pore label to a nucleotide label, which results in emission of lower energy radiation. The nucleotide label radiation is then detected by a confocal microscope setup or other optical detection system or light microscopy system capable of single molecule detection known to people having ordinary skill in the art. Examples of such detection systems include but are not limited to confocal microscopy, epifluorescent microscopy and total internal reflection fluorescent (TIRF) microscopy. Other polymers (e.g., proteins and polymers other than nucleic acids) having labeled monomers may also be sequenced according to the methods described herein.

Energy may be transferred from a pore or nanopore donor label (e.g., a Quantum Dot) to an acceptor label on a polymer (e.g., a nucleic acid) when an acceptor label of an acceptor labeled monomer (e.g., nucleotide) of the polymer interacts with the donor label as, after or before the labeled monomer exits, enters or passes through a nanopore. For example, the donor label may be positioned on or attached to the nanopore on the cis or trans side or surface of the nanopore such that the interaction or energy transfer between the donor label and acceptor label does not take place until the labeled monomer exits the nanopore and comes into the vicinity or proximity of the donor label outside of the nanopore channel or opening. As a result, interaction between the labels, energy transfer from the donor label to the acceptor label, emission of energy from the acceptor label and/or measurement or detection of an emission of energy from the acceptor label may take place outside of the passage, channel or opening running through the nanopore, e.g., within a cis or trans chamber on the cis or trans sides of a nanopore. The measurement or detection of the energy emitted from the acceptor label of a monomer may be utilized to identify the monomer.

The nanopore label may be positioned outside of the passage, channel or opening of the nanopore such that the label may be visible or exposed to facilitate excitation or illumination of the label. The interaction and energy transfer between a donor label and acceptor label and the emission of energy from the acceptor label as a result of the energy transfer may take place outside of the passage, channel or opening of the nanopore. This may facilitate ease and accuracy of the detection or measurement of energy or light emission from the acceptor label, e.g., via an optical detection or measurement device. The donor and acceptor label interaction may take place within a channel of a nanopore and a donor label could be positioned within the channel of a nanopore.

A donor label may be attached in various manners and/or at various sites on a nanopore. For example, a donor label may be directly or indirectly attached or connected to a portion or unit of the nanopore. Alternatively, a donor label may be positioned adjacent to a nanopore.

Each acceptor labeled monomer (e.g., nucleotide) of a polymer (e.g., nucleic acid) can interact sequentially with a donor label positioned on or next to or attached directly or indirectly to a nanopore or channel through which the polymer is translocated. The interaction between the donor and acceptor labels may take place outside of the nanopore channel or opening, e.g., after the acceptor labeled monomer exits the nanopore or before the monomer enters the nanopore. The interaction may take place within or partially within the nanopore channel or opening, e.g., while the acceptor labeled monomer passes through, enters or exits the nanopore.

When one of the four nucleotides of a nucleic acid is labeled, the time dependent signal arising from the single nucleotide label emission can be converted into a sequence corresponding to the positions of the labeled nucleotide in the nucleic acid sequence. The process can then be repeated for each of the four nucleotides in separate samples and the four partial sequences are then aligned to assemble an entire nucleic acid sequence.

When multi-color labeled nucleic acid (DNA) sequences are analyzed, the energy transfer from one or more donor labels to each of the four distinct acceptor labels that may exist on a nucleic acid molecule may result in light emission at four distinct wavelengths or colors (each associated with one of the four nucleotides) which can allow for a direct sequence read-out.

During sequencing of a nucleic acid molecule, the energy transfer signal may be generated with sufficient intensity that a sensitive detection system can accumulate sufficient signal within the transit time of a single nucleotide through the nanopore to distinguish a labeled nucleotide from an unlabeled nucleotide. Therefore, the pore label may be stable, have a high absorption cross-section, a short excited state lifetime, and/or temporally homogeneous excitation and energy transfer properties. The nucleotide label may be capable of emitting and absorbing sufficient radiation to be detected during the transit time of the nucleotide through the pore. The product of the energy transfer cross-section, emission rate, and quantum yield of emission may yield sufficient radiation intensity for detection within the single nucleotide transit time. A nucleotide label may also be sufficiently stable to emit the required radiation intensity and without transience in radiation emission.

The excitation radiation source may be of high enough intensity that when focused to the diffraction limit on the nanopore, the radiation flux is sufficient to saturate the pore label. The detection system may filter out excitation radiation and pore label emission while capturing nucleic acid label emission during pore transit with sufficient signal-to-noise ratio (S/N) to distinguish a labeled nucleotide from an unlabeled nucleotide with high certainty. The collected nucleic acid label radiation may be counted over an integration time equivalent to the single nucleotide pore transit time.

A software signal analysis algorithm may then be utilized which converts the binned radiation intensity signal to a sequence corresponding to a particular nucleotide. Combination and alignment of four individual nucleotide sequences (where one of the four nucleotides in each sequence is labeled) allows construction of the complete nucleic acid sequence via a specifically designed computer algorithm.

A system for sequencing one or more biological polymers, e.g., nucleic acid molecules, may include a fixture or pore holder. The pore holder may include a nanopore membrane assembly wherein one or more nanopores span a lipid bilayer membrane. The nanopore membrane assembly has a Cis (−) side and a Trans (+) side. One or more labels may be attached to the nanopores. Alternatively, a label may be attached to a portion of the membrane or substrate through which the nanopore spans or to a linker or other molecule attached to the membrane, substrate or nanopore. An aqueous buffer solution is provided which surrounds the nanopore membrane assembly. The pore holder may contain two electrodes. A negative electrode or terminal may be positioned on the Cis side of the nanopore membrane assembly and a positive electrode or terminal may be positioned on the Trans side of the nanopore membrane assembly.

A flow of fluid or solution is provided on the side of the nanopore where the translocated polymer or nucleic acid exits after translocation through the nanopore. The flow may be continuous or constant such that the fluid or solution does not remain static for an extended period of time. The fluid flow or motion helps move or transfer translocated polymers away from the nanopore channel such the translocated polymers do not linger or accumulated near the nanopore channel exit or opening and cause fluorescent background or noise which could disrupt or prevent an accurate reading, measurement or detection of the energy emitted by a polymer acceptor label. Translocated polymers may include labels that were not fully exhausted, i.e., they have not reached their fluorescent lifetime and are still able to emit light. Such labels could interfere with the energy transfer between donor labels and subsequent monomer labels or emit energy that may interfere with the emission from other labels and disrupt an accurate reading or detection of energy from a labeled monomer.

One or more polymers, e.g., nucleic acid polymers or molecules, to be analyzed may also be provided. A polymer or nucleic acid polymer or molecule may include one or more labels, e.g., one or more monomers or nucleotides of the polymer may be labeled. A nucleic acid molecule may be loaded into a port positioned on the Cis side of then nanopore membrane assembly. The membrane segregates the nucleic acids to be analyzed to the Cis side of the nanopore membrane assembly. An energy source, e.g., an illumination source, for exciting the nanopore label may be utilized. An electric field may be applied to or by the electrodes to force the labeled nucleic acid to translocate through the nanopore into the Cis side and out of the Trans side of the nanopore, from the Cis to the Trans side of the membrane, e.g., in a single file (Kasianowicz, J. J. et al., Proc. Natl. Acad. Sci. USA 93 (1996): 13770-13773). Optionally, an electrical field may be applied utilizing other mechanisms to force the labeled nucleic acid to translocate through the nanopore. When a nucleic acid molecule is translocated through the nanopore and a labeled nucleotide comes into close proximity with the nanopore label, e.g., upon or after exiting the nanopore, energy is transferred from the excited nanopore label to a nucleotide label. A detector or detection system, e.g., optical detection system, for detecting or measuring energy emitted from the nucleotide label as a result of the transfer of energy from the nanopore label to the nucleotide label may also be provided.

The pore may be labeled with one or more donor labels in the form of quantum dots, metal nanoparticles, nano diamonds or fluorophores. The pore may be illuminated by monochromatic laser radiation. The monochromatic laser radiation may be focused to a diffraction limited spot exciting the quantum dot pore labels. As the labeled nucleic acid (e.g., labeled with an acceptor label in the form of a fluorophore) is translocated through the nanopore, the pore donor label (also “pore label” or “donor label”) and a nucleotide acceptor label come into close proximity with one another and participate in a FRET (Förster resonance energy transfer) energy exchange interaction between the pore donor label and nucleic acid acceptor label (Ha, T. et al., Proc. Natl. Acad. Sci. USA 93 (1996): 6264-6268).

FRET is a non-radiative dipole-dipole energy transfer mechanism from a donor to acceptor fluorophore. The efficiency of FRET may be dependent upon the distance between donor and acceptor as well as the properties of the fluorophores (Stryer, L., Annu Rev Biochem. 47 (1978): 819-846).

A fluorophore may be any construct that is capable of absorbing light of a given energy and re-emitting that light at a different energy. Fluorophores include, e.g., organic molecules, rare-earth ions, metal nanoparticles, nanodiamonds and semiconductor quantum dots.

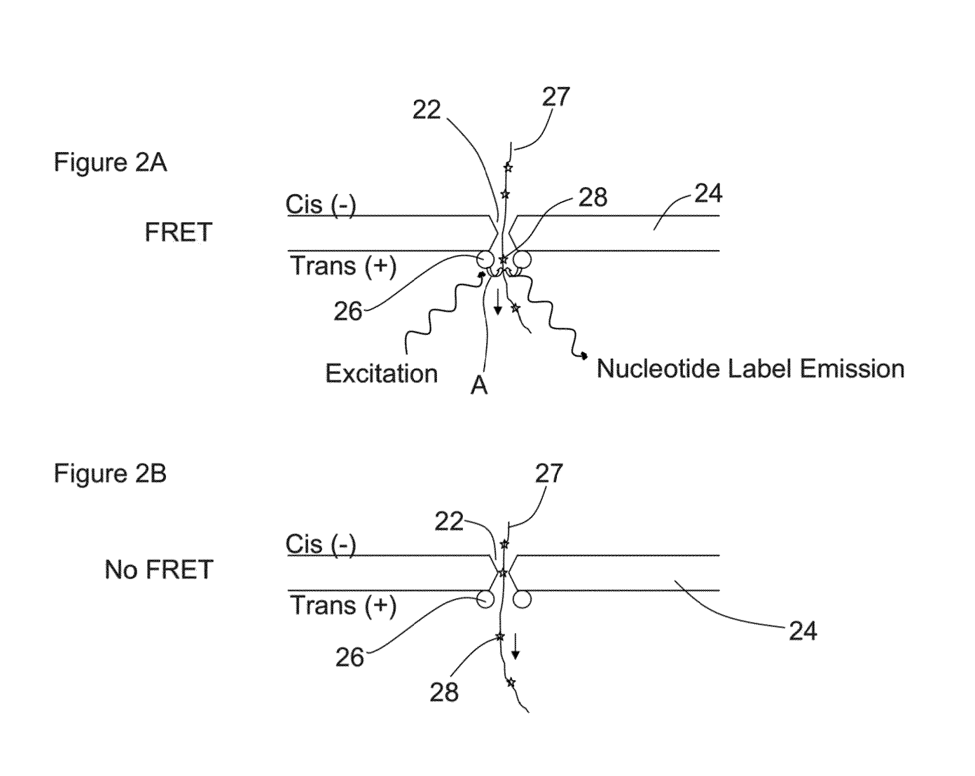

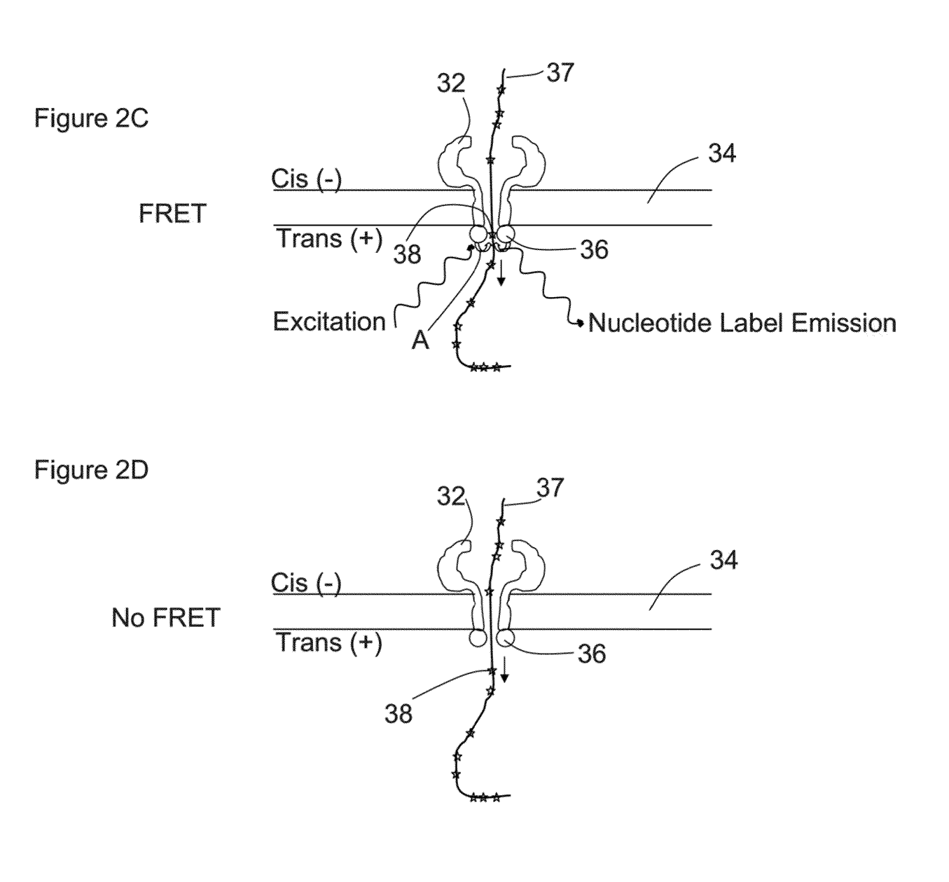

FIG. 2A shows one variation of a FRET interaction between a pore donor label 26 on a synthetic nanopore 22 and a nucleic acid acceptor label 28 on a nucleic acid 27 (e.g., a single or double stranded nucleic acid), which is being translocated through the synthetic nanopore 22. The synthetic nanopore 22 is positioned in a substrate 24. FRET is a non-radiative dipole-dipole energy transfer mechanism from a donor label 26 to an acceptor label 28 (e.g., a fluorophore). The efficiency of the energy transfer is, among other variables, dependent on the physical distance between acceptor label 28 and the donor label.

The nucleic acid acceptor label 28 positioned on a nucleotide of the nucleic acid moves into close proximity with an excited nanopore donor label 26, e.g., as or after the label 28 or labeled nucleotide exits the nanopore 22, and gets excited via FRET (indicated by the arrow A showing energy transfer from the pore label 26 to the nucleic acid label 28). As a result, the nucleic acid label 28 emits light of a specific wavelength, which can then be detected with the appropriate optical equipment or detection system in order to identify the labeled nucleotide corresponding to or associated with the detected wavelength of emitted light.

FIG. 2B shows translocation of the labeled nucleic acid 27 at a point in time where no FRET is taking place (due to the acceptor and donor labels not being in close enough proximity to each other). This is indicated by the lack of any arrows showing energy transfer between a pore label 26 and a nucleic acid label 28.

FIG. 2C shows one variation of a FRET interaction between a pore donor label 36 on a proteinaceous or protein nanopore 32 and a nucleic acid acceptor label 38 on a nucleic acid 37 (e.g., a single or double stranded nucleic acid), which is being translocated through the protein pore or nanopore 32. The pore protein 32 is positioned in a lipid bilayer 34. The nucleic acid acceptor label 38 positioned on a nucleotide of the nucleic acid moves into close proximity with an excited nanopore donor label 36, e.g., as or after the label 38 or labeled nucleotide exits the nanopore 32, and gets excited via FRET (indicated by the arrow A showing energy transfer from the pore label 36 to the nucleic acid label 38). As a result, the nucleic acid label 38 emits light of a specific wavelength, which can be detected with the appropriate optical equipment or detection system in order to identify the labeled nucleotide corresponding to or associated with the detected wavelength of emitted light.

FIG. 2D shows translocation of the labeled nucleic acid 37 at a point in time where no FRET is taking place (due to the labels not being in close enough proximity to each other). This is indicated by the lack of arrows showing energy transfer between a pore donor label 36 and a nucleic acid label 38.

Three equations are also shown below: Equation (1) gives the Förster radius which is defined as the distance that energy transfer efficiency from donor to acceptor is 50%. The Förster distance depends on the refractive index (n_{D}), quantum yield of the donor (Q_{D}), spatial orientation (K) and the spectral overlap of the acceptor and donor spectrum (I). N_{A} is the Avogadro number with N_{A}=6.022×10²³ mol⁻¹ (see equation below). Equation (2) describes the overlap integral for the donor and acceptor emission and absorption spectra respectively; Equation (3) shows the FRET energy transfer efficiency as a function of distance between the acceptor and donor pair. The equations demonstrate that spectral overlap controls the Förster radius, which determines the energy transfer efficiency for a given distance between the FRET pair. Therefore by tuning the emission wavelength of the donor, the distance at which energy transfer occurs can be controlled.

$$\begin{matrix}{R}_{0} = {( \frac{9000 ( ln 10 ) {κ}_{p}^{2} {Q}_{D}}{{N}_{A} 128 {π}^{5} {n}_{D}^{4}} l )}^{1 / 6} & ( 1 ) \\ l = \int J ( λ ) d λ = \int {PL}_{D - corr} ( λ ) \times {λ}^{4} \times {ɛ}_{A} ( λ ) d λ & ( 2 ) \\ E = \frac{{k}_{DA}}{{k}_{DA} + {τ}_{D}^{- 1}} = \frac{{R}_{0}^{6}}{{R}_{0}^{6} + {r}^{6}} & ( 3 )\end{matrix}$$

With respect to Quantum dots, due to the size dependent optical properties of quantum dots, the donor emission wavelength may be adjusted. This allows the spectral overlap between donor emission and acceptor absorption to be adjusted so that the Förster radius for the FRET pair may be controlled. The emission spectrum for Quantum dots is narrow, (e.g., 25 nm Full width-half maximum—FWHM—is typical for individual quantum dots) and the emission wavelength is adjustable by size, enabling control over the donor label-acceptor label interaction distance by changing the size of the quantum dots. Another important attribute of quantum dots is their broad absorption spectrum, which allows them to be excited at energies that do not directly excite the acceptor label. The properties allow quantum dots of the properly chosen size to be used to efficiently transfer energy with sufficient resolution to excite individual labeled nucleotides as, after or before the labeled nucleotides travel through a donor labeled pore.

Following a FRET energy transfer, the pore donor label may return to the electronic ground state and the nucleotide acceptor label can re-emit radiation at a lower energy. Where fluorophore labeled nucleotides are utilized, energy transferred from the fluorophore acceptor label results in emitted photons of the acceptor label. The emitted photons of the acceptor label may exhibit lower energy than the pore label emission. The detection system for fluorescent nucleotide labels may be designed to collect the maximum number of photons at the acceptor label emission wavelength while filtering out emission from a donor label (e.g., quantum dot donors) and laser excitation. The detection system counts photons from the labeled monomers as a function of time. Photon counts are binned into time intervals corresponding to the translocation time of, for instance, a monomer comprising a single nucleotide in a nucleic acid polymer crossing the nanopore. Spikes in photon counts correspond to labeled nucleotides translocating across the pore. To sequence the nucleic acid, sequence information for a given nucleotide is determined by the pattern of spikes in photon counts as a function of time. An increase in photon counts is interpreted as a labeled nucleotide.

Translocation of nucleic acid polymers through the nanopore may be monitored by current measurements arising from the flow of ions through the nanopore. Translocating nucleic acids partially block the ionic flux through the pore resulting in a measurable drop in current. Thus, detection of a current drop represents detection of a nucleic acid entering the pore, and recovery of the current to the original value represents detection of a nucleic acid exiting the pore.

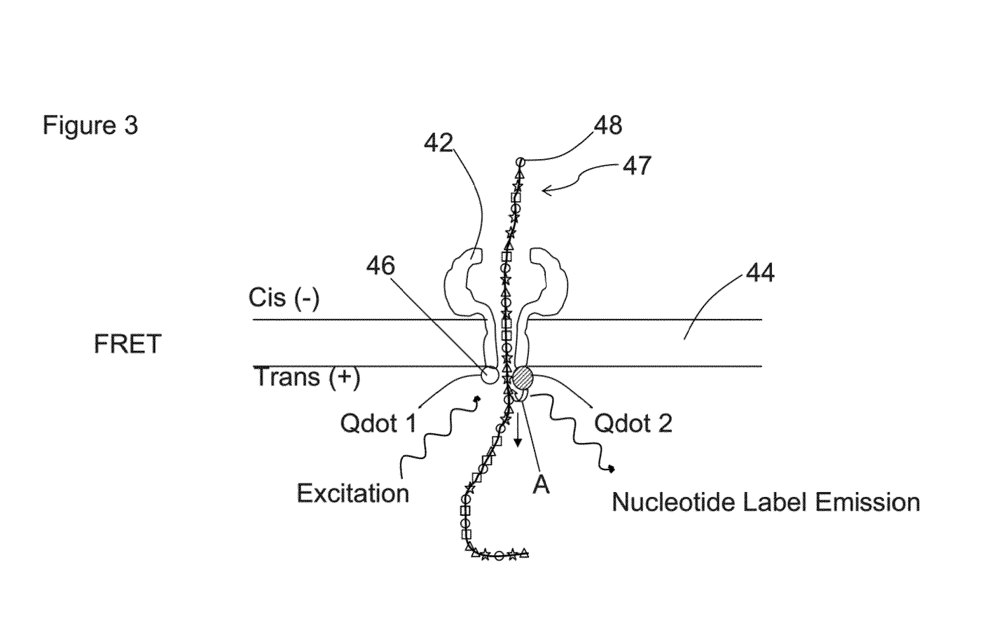

As mentioned supra, a multicolor FRET interaction is utilized to sequence a molecule such a nucleic acid. FIG. 3 shows one variation of a multicolor FRET interaction between one or more donor labels 46 (e.g., Quantum dots) of a protein nanopore 42 (optionally, a synthetic nanopore may be utilized) and one or more acceptor labels 48 of a nucleic acid molecule 47 (e.g., a single or double stranded nucleic acid). Each shape on the nucleic acid 47 represents a specific type of acceptor label labeling a nucleotide, where each label has a distinct emission spectrum associated with or corresponding to a specific nucleotide such that each label emits light at a specific wavelength or color associated with a specific nucleotide.

In FIG. 3, each of the four shapes (triangle, rectangle, star, circle) represents a specific acceptor label 48, each label having a distinct emission spectra (e.g., 4 different emission spectra). Each of the acceptor labels 48 can form a FRET pair with a corresponding donor label or quantum dot 46 attached to the base of the nanopore. Qdot1 and Qdot2 represent two different Quantum dots as donor labels 46 that form specific FRET pairs with a nucleic acid acceptor label 48. The Quantum dot donor labels 46 are in an excited state and depending on the particular acceptor label 48 that comes in proximity to the Quantum dots during, after or before a labeled nucleotide translocation through the nanopore 42, an energy transfer (arrow A) from the donor label 46 to the nucleotide acceptor label 48 takes place, resulting in a nucleotide label 48 energy emission. As a result, each nucleotide may emit light at a specific wavelength or color (due to the distinct emission spectrum of the nucleotide's label), which can be detected (e.g., by optical detection) and used to identify or deduce the nucleotide sequence of the nucleic acid 47 and the nucleic acid 47 sequence.

Different pore labels exhibiting different spectral absorption maxima may be attached to a single pore. The nucleic acid may be modified with corresponding acceptor dye labeled nucleotides where each donor label forms FRET pairs with one acceptor labeled nucleotide (i.e. multi-color FRET). Each of the four nucleotides may contain a specific acceptor label which gets excited by one or more of the pore donor labels. The base of the pore may be illuminated with different color light sources to accommodate the excitation of the different donor labels. Alternatively, e.g., where Quantum dots are used as donor labels, the broad absorption spectra characteristic of Quantum dots may allow for a single wavelength light source to sufficiently illuminate/excitate the different donor labels which exhibit different spectral absorption maxima.

A single pore donor label (e.g., a single Quantum dot) may be suitable for exciting one nucleic acid acceptor label. For example, four different pore donor labels may be provided where each donor label can excite one of four different nucleic acid acceptor labels resulting in the emission of four distinct wavelengths. A single pore donor label (e.g., a single Quantum dot) may be suitable for exciting two or more nucleic acid acceptor labels that have similar absorption spectra overlapping with the donor label emission spectrum and show different emission spectra (i.e. different Stoke's shifts), where each acceptor label emits light at a different wavelength after excitation by the single donor label. Two different pore donor labels (e.g., two Quantum dots having different emission or absorption spectra) may be suitable for exciting four nucleic acid acceptor labels having different emission or excitation spectra, which each emit light at different wavelengths. One donor label or Quantum dot may be capable of exciting two of the nucleic acid acceptor labels resulting in their emission of light at different wavelengths, and the other Quantum dot may be capable of exciting the other two nucleic acid acceptor labels resulting in their emission of light at different wavelengths. The above arrangements provide clean and distinct wavelength emissions from each nucleic acid acceptor label for accurate detection.

A nanopore may include one or more monomers or attachment points, e.g., about 7 attachment points, one on each of the seven monomers making up a particular protein nanopore, such as alpha-hemolysin. One or more different donor labels, e.g., Quantum dots, may attach one to each of the attachment points, e.g., a nanopore may have up to seven different Quantum dots attached thereto. A single donor label or Quantum dot may be used to excite all four different nucleic acid acceptor labels resulting in a common wavelength emission suitable for detecting a molecule or detecting the presence of a molecule, e.g., in a biosensor application.

For accumulation of the raw signal data where a multi-color FRET interaction is utilized, the emission wavelength of the four different acceptor labels may be filtered and recorded as a function of time and emission wavelength, which results in a direct read-out of sequence information.

As mentioned supra, a nucleic acid sample may be divided into four parts to sequence the nucleic acid. The four nucleic acid or DNA samples may be used as a template to synthesize a labeled complementary nucleic acid polymer. Each of the four nucleic acid samples may be converted in a way such that one of the four nucleotide types (Guanine, Adenine, Cytosine or Thymine) are replaced with the nucleotide's labeled counterpart or otherwise labeled by attaching a label to a respective nucleotide. The same label may be used for each nucleotide or optionally, different labels may be used. The remaining nucleotides are the naturally occurring nucleic acid building blocks. Optionally, two, three or each nucleotide of a nucleic acid may be replaced with a nucleotide carrying a distinct acceptor label.

To perform the sequence read-out where a single nucleotide label is utilized with the target nucleic acid split into four samples, each having one nucleotide labeled with the same, or optionally, a different acceptor label, a specially designed algorithm may be utilized which (i) corrects, (ii) defines, and (iii) aligns the four partial sequences into one master sequence. Each partial sequence displays the relative position of one of the four nucleotides in the context of the whole genome sequence, thus, four sequencing reactions may be required to determine the position of each nucleotide.

The algorithm may correct for missing bases due to inefficient labeling of the nucleic acid. One type of nucleotide in a DNA molecule can be completely substituted with the nucleotide's fluorescent counterpart. Various inefficiencies in labeling may result in less than 100% coverage from this substitution. Fluorescently labeled nucleotides usually come at a purity of around 99%, i.e., approximately 1% of the nucleotides do not carry a label. Consequently, even at a 100% incorporation of modified nucleotides, 1% of the nucleotides may be unlabeled and may not be detectable by nanopore transfer sequencing.

One solution to this problem is a redundant coverage of the nucleic acid to be sequenced. Each sequence may be read multiple times, e.g., at least 50 times per sequencing reaction (i.e. a 50 fold redundancy). Thus, the algorithm will compare the 50 sequences which will allow a statistically sound determination of each nucleotide call.

The algorithm may define the relative position of the sequenced nucleotides in the template nucleic acid. For example, the time of the current blockage during the translocation process may be used to determine the relative position of the detected nucleotides. The relative position and the time of the occurrence of two signals may be monitored and used to determine the position of the nucleotides relative to each other. Optionally, a combination of the above methods may be used to determine the position of the nucleotides in the sequence.

The nucleic acid or DNA to be analyzed may be separated into four samples. Each sample will be used to exchange one form of nucleotide (A, G, T, or C) with the nucleotide's fluorescent counterpart. Four separate nanopore sequencing reactions may reveal the relative positions of the four nucleotides in the DNA sample through optical detection. A computer algorithm will then align the four sub-sequences into one master sequence. The same acceptor label capable of emitting light at a specific wavelength or color may be utilized in all four samples. Optionally, different labels having different wavelength emissions may be utilized.

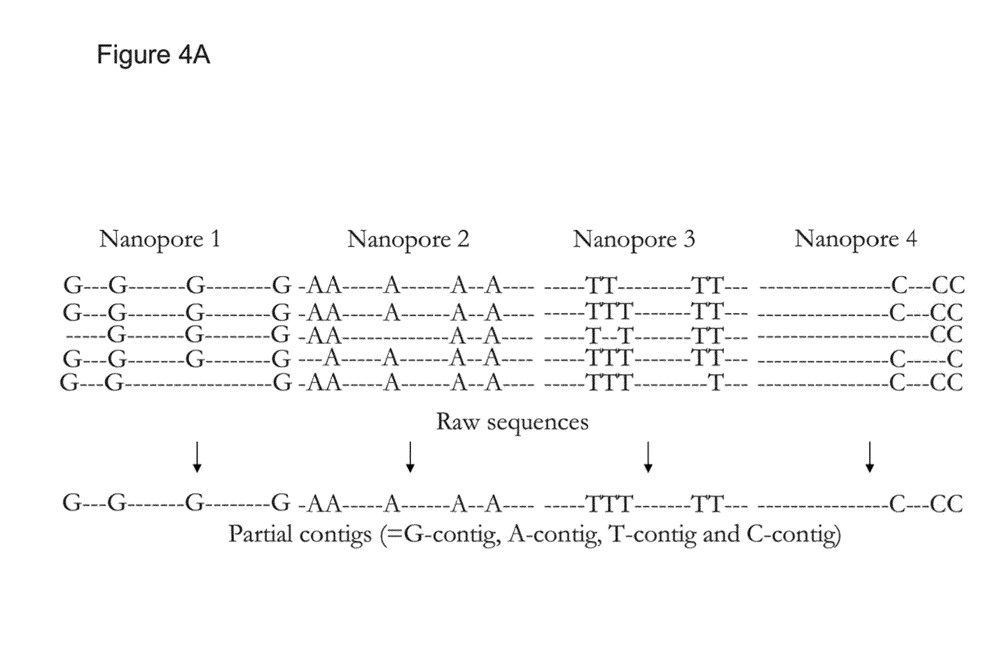

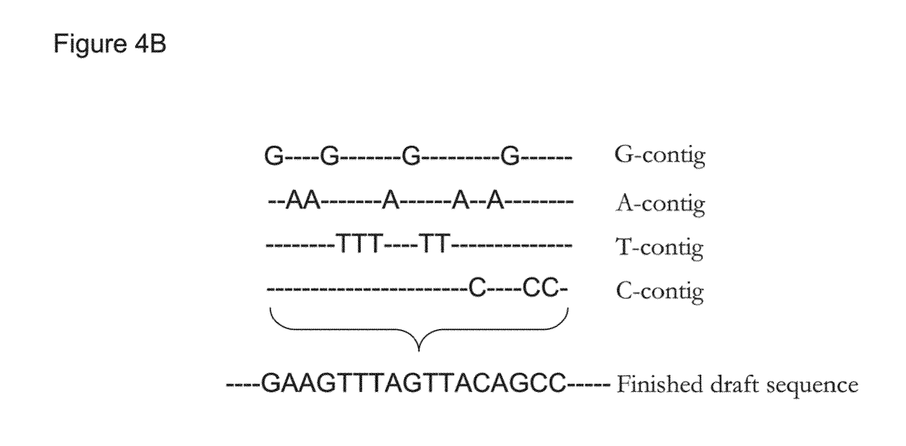

For example, FIG. 4A shows partial contigs from nucleic acid sequencing utilizing a singly labeled nucleic acid. Four separate nanopore sequencing reactions take place. Each of the four separate nanopore sequencing reactions, which are created by the same type of nucleotide acceptor label, generates a sub-sequence that displays the relative position of one of the four nucleotides. A redundant coverage of each sequence may ensure statistical sound base calls and read-outs. A computer algorithm may be utilized to deduce the four partial contig sequences which are the common denominators of the multiple covered sub-sequences (i.e. G-contig, A-contig, T-contig, and C-contig).

FIG. 4B shows how partial contig alignment may generate a first draft nucleic acid sequence. For example, the second bioinformatic step involves alignment of the four contigs. Software searches for matching sequence stretches of the four contigs that complement each other. This step results in a finished draft sequence.

Optionally, both optical and electrical read-outs/detection may be utilized to sequence a nucleic acid. Electrical read-outs may be utilized to measure the number of non-labeled nucleotides in a sequence to help assess the relative position of a detected labeled nucleotide on a nucleic acid sequence. The length of the nucleic acid can be calculated by measuring the change in current through the nanopore and the duration of that current change. The methods and systems described herein may utilize solely optical read-outs or optical detection of energy emission or light emission by a labeled monomer to identify and sequence the monomer and to sequence a polymer including the monomer. Optionally, a combination of optical and electrical readouts or detection may be used.

A nucleotide acceptor label may be in the form of a quencher which may quench the transferred energy. In the case of a quenching nucleotide label, radiation emission from the pore donor label will decrease when a labeled nucleotide is in proximity to the donor label. The detection system for quenching pore labels is designed to maximize the radiation collected from the pore labels, while filtering out laser excitation radiation. For a quenching label, a decrease in photon counts of the pore label, such as a quantum dot, is interpreted as a labeled nucleotide.

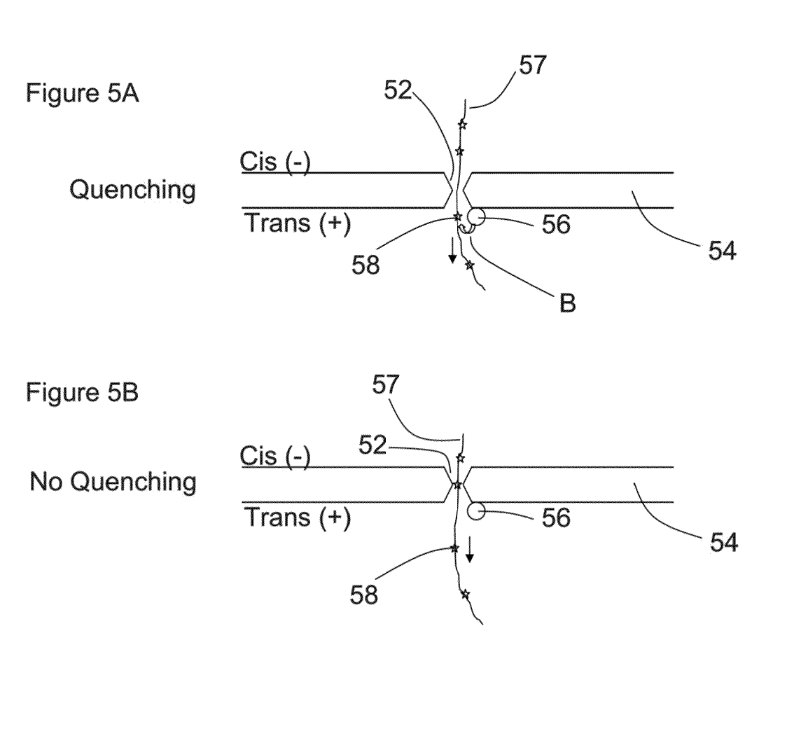

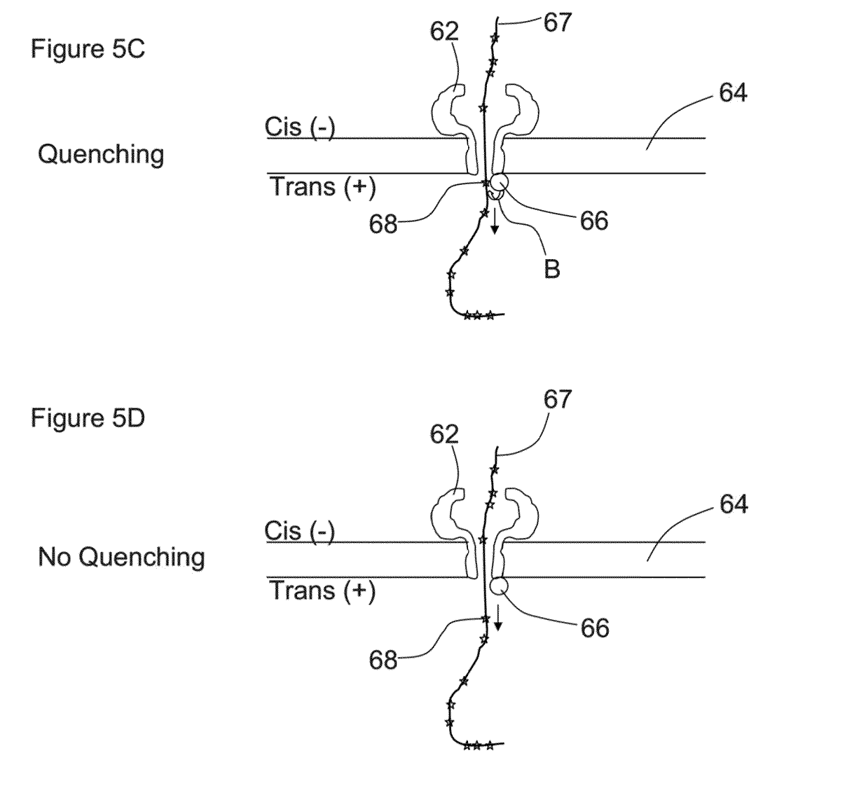

FIG. 5A shows one variation of a quenching interaction between a pore donor label 56 on a synthetic pore or nanopore 52 and a nucleic acid quenching label 58 on a nucleic acid 57 (e.g., a single or double stranded nucleic acid), which is being translocated through the synthetic nanopore 52. The synthetic nanopore 52 is positioned in a substrate 54, e.g., a solid substrate.

During a continuous or substantially continuous illumination of the pore label 56, the pore label 56 emits light at a certain wavelength which is detected with an appropriate optical or other detection system. The quenching label 58 positioned on a nucleotide of nucleic acid 57 comes in close proximity to the pore label 56, e.g., as or after the label 58 or labeled nucleotide exits the nanopore 52, and thereby quenches the pore label 56. The quenching label 58 acts by absorption of energy from the illuminated pore label 56 (which is indicated by arrow B) causing the photon emission from the pore label 56 to change. For example, the quenching may be detected by detecting a change, such as a decrease or diminishing, in the photons emitted by the nanopore label. A degree of photon emission change may be associated with or correspond to a single nucleotide of the nucleic acid molecule and as such, the nucleic acid molecule sequence may be deduced based on detecting the change in photon emission by the donor label caused by the quenching label.

FIG. 5B shows translocation of the labeled nucleic acid 57 at a point in time where no quenching is taking place (due to the labels not being in close enough proximity to each other). This is indicated by the lack of any arrows showing energy transfer between a pore label 56 and a nucleic acid label 58.

FIG. 5C shows one variation of a quenching interaction between a pore donor label 66 on a proteinaceous or protein pore or nanopore 62 and a nucleic acid quenching label 68 on a nucleic acid 67 (e.g., a single or double stranded nucleic acid), which is being translocated through the protein nanopore 62. The protein nanopore 62 is positioned in a lipid bilayer 64.

During a continuous illumination of the pore label 66 the pore label 66 emits light at a certain wavelength which is detected with an appropriate optical or other detection system. The quenching label 68 positioned on a nucleotide of nucleic acid 67 comes in close proximity to the pore label 66, e.g., as or after the label 68 or labeled nucleotide exits the nanopore 62, and thereby quenches the pore label 66 (which is indicated by arrow B). This quenching is detected by a decrease or sharp decrease in measured photons emitted from the nanopore label.

FIG. 5D shows translocation of the labeled nucleic acid 67 at a point in time where no quenching is taking place (due to the labels not being in close enough proximity to each other). This is indicated by the lack of any arrows showing energy transfer between a pore label 66 and a nucleic acid label 68.

The energy transfer reaction, energy emission or pore label quenching as described above may take place as or before the label or labeled nucleotide enters the nanopore, e.g., on the cis side of the nanopore.

The labeling system may be designed to emit energy continuously without intermittency or rapid photobleaching of the fluorophores. For example, the buffer compartment of a pore holder may contain an oxygen depletion system that will remove dissolved Oxygen from the system via enzymatical, chemical or electrochemical means thereby reducing photobleaching of the fluorophore labeled nucleic acid.

An oxygen depletion system is a buffer solution containing components that selectively react with dissolved oxygen. Removing oxygen from the sequencing buffer solution helps prevent photobleaching of the fluorophore labels. An example of a composition of an oxygen depletion buffer is as follows: 10 mM tris-Cl, pH 8.0, 50 mM NaCl, 10 mM MgCl2, 1% (v/v) 2-mercaptoethanol, 4 mg/ml glucose, 0.1 mg/ml glucose oxidase, and 0.04 mg/ml catalase (Sabanayagam, C. R. et al., J. Chem. Phys. 123 (2005): 224708). The buffer is degassed by sonication before use to extend the buffer's useful lifetime by first removing oxygen mechanically. The buffer system then removes oxygen via the enzymatic oxidation of glucose by glucose oxidase.

The sequencing buffer may also contain components that prevent fluorescence intermittency, also referred to as “blinking,” in one or both of the quantum dot labeled pores and fluorophore labeled nucleic acids. The phenomenon of blinking occurs when the excited fluorophore transitions to a non-radiative triplet state. Individual fluorophores may display fluorescence intermittency known as blinking in which the fluorophore transitions to and from the fluorophore's emitting and dark state. Blinking can interfere with certain aspects of the sequencing schemes. Blinking may be prevented or left alone. The triplet state is responsible for blinking in many organic fluorophores and that blinking can be suppressed with chemicals that quench the triplet state.

Molecules such as Trolox (6-hydroxy-2,5,7,8-tetramethylchroman-2-carboxylic acid) are effective in eliminating blinking for fluorophores or dyes such as Cy5 (Rasnik, I. et al., Nat Methods 11 (2006): 891-893). Certain Quantum dots may display blinking, however, CdTe quantum dots produced by aqueous synthesis in the presence of mercaptopropionic acid have recently been shown to emit continuously without blinking (He, H. et al., Angew. Chem. Int. Ed. 45 (2006): 7588-7591). CdTe quantum dots are ideally suited as labels to be utilized in the methods described herein, since they are water soluble with high quantum yield and can be directly conjugated through the terminal carboxylic acid groups of the mercaptopropionic acid ligands.

The labels may be made resistant to photobleaching and blinking. With an efficient oxygen depletion system, Cy5 fluorophores can undergo ˜10̂5 cycles of excitation and emission before irreversible degradation. If the incident laser light is of high enough efficiency that excitation of the Cy5 fluorophore is saturated (re-excited immediately after emission) than the rate of photon emission is determined by the fluorescence lifetime of the Cy5 fluorophore. Since the Cy5 fluorophore has a lifetime on the order of Ins, and an assumed FRET efficiency of 10%, up to 10,000 photons can be emitted as the Cy5 labeled nucleotide transverses the nanopore. Microscopes used for single molecule detection are typically around 3% efficient in light collection. This can provide ˜300 photons detected for a given label, which provides sufficiently high signal to noise ratio for single base detection.

A labeled polymer or nucleic acid may be translocated through a nanopore having a suitable diameter (the diameter may vary, e.g., the diameter may be about 2 to 6 nm) at an approx. speed of 1,000 to 100,000 or 1,000 to 10,000 nucleotides per second. Each base of the nucleic acid may be fluorescently labeled with a distinct fluorophore. The base of the nanopore may be labeled with a quantum dot. When the nucleotide label comes in close proximity to the quantum dot, a non-radiative, quantum resonance energy transfer occurs which results in light emission of a specific wavelength form the nucleotide label.

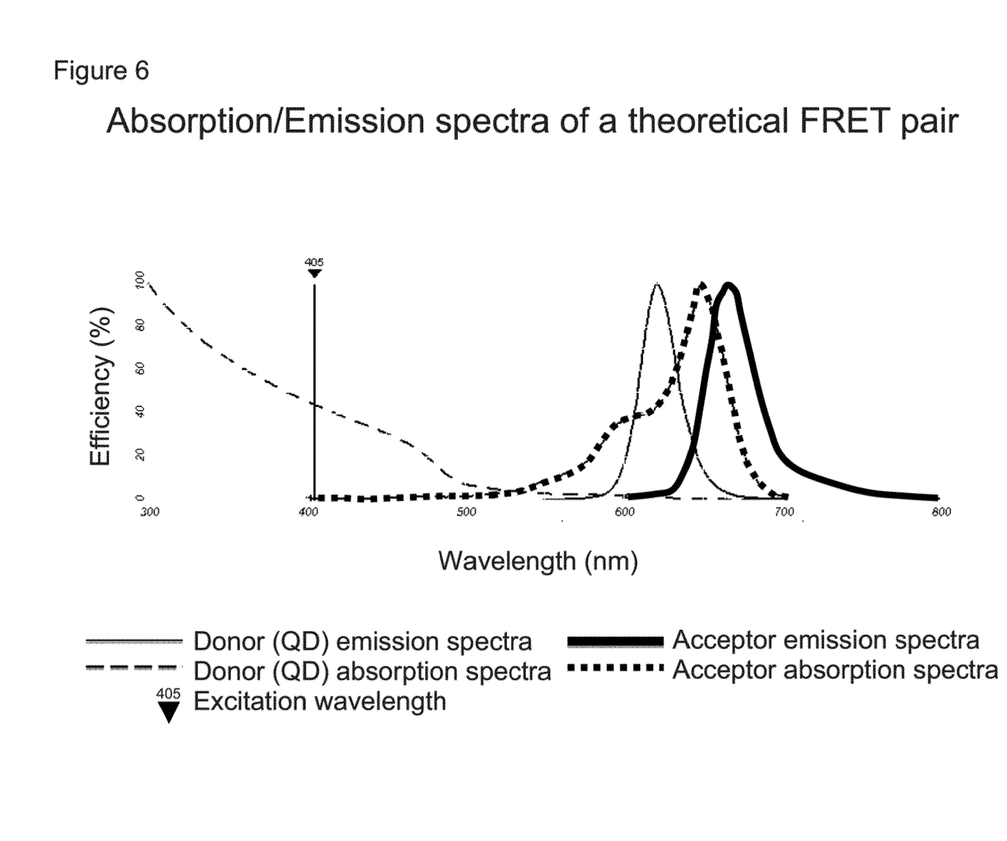

FIG. 6 shows an example of an absorption/emission spectra from a FRET pair containing a donor quantum dot and an acceptor fluorophore. The characteristic broad absorption peak (thin dashed line) of the quantum dot allows for a short excitation wavelength which does not interfere with the detection of the longer emission wavelength. The emission peak of the quantum dot (thin solid line) has a significant spectral overlap with the absorption peak of the acceptor fluorophore (thick dashed line). This overlap may result in an energy transfer from the quantum dot to the fluorophore which then emits photons of a specific wavelength (thick solid line). These fluorophore emitted photons are subsequently detected by an appropriate optical system. The efficiency of the energy transfer may be highly dependent on the distance between the donor and acceptor, with a 50% efficiency at the so called Foerster radius.

Sequencing may be performed by utilizing one or more pores or nanopores simultaneously. For example, a plurality of nanopores may be positioned in parallel or in any configuration in one or more lipid bilayers or substrates in order to expedite the sequencing process and sequence many nucleic acid molecules or other biological polymers at the same time.

A plurality of pores may be configured on a rotatable disc or substrate. When donor labels or quantum dots become substantially or completely used, burned out or exhausted (i.e., they reached their fluorescent lifetime), the disc or substrate may be rotated, thereby rotating a fresh pore with fresh donor labels or quantum dots into place to receive nucleic acids and continue sequencing. The electrical field which pulls the nucleic acid through the pore may be turned off during rotation of the disc and then turned back on once a new pore is in position for sequencing. Optionally, the electric field may be left on continuously.

In certain variations, various detection methods and systems, e.g., a biosensor, for detecting or identifying molecules or performing polymer sequencing using an opening, pore, nanopore, channel or passage, e.g., an ion permeable pore, nanopore, channel or passage are provided. These detection methods or systems may utilize the positional sensitivity of Förster Resonance Energy Transfer (FRET) between a donor on a channel or nanopore, e.g., a fluorescent donor, and an acceptor on a molecule, e.g., an acceptor dye. The detection may be optical detection.

A nanopore, pore, passage or channel may include any opening positioned in a substrate that allows the passage of a molecule through the substrate. For example, the nanopore or pore may allow passage of a molecule that would otherwise not be able to pass through that substrate. Examples of nanopores or pores include proteinaceous or protein based pores or synthetic pores. In certain variations, a nanopore may have an inner diameter of 1-10 nm or 1-5 nm or 1-3 nm (nanometers).

In certain variations, a nanopore, pore, passage or channel may include any orifice, opening, or through hole in a substrate that only allows the single file passage of a molecule therethrough. For example, a nanopore, pore or channel that is used to sequence a DNA molecule may have a diameter that does not exceed 2 nm (for single stranded DNA) or up to 3 nm for double stranded DNA. Similar dimensions may be utilized for other polymers or molecules. In certain variations, a nanopore, pore, channel, orifice or through hole in a substrate may have a diameter of about 1 to 10 nm or about 1 to 5 nm or about 1-3 nm which allows only the single file passage of molecules. In certain variations, the nanopores, pores or channels may be of a size to allow for sequential molecule by molecule or monomer by monomer transfer therethrough of only one molecule or polymer at a time.

In certain variations, a method of detecting a molecule, for example, a method of sequencing a polymer, e.g., a nucleic acid, may include one or more of the following steps. One or more pores, nanopores or channels may be provided on a substrate. A polymer may be translocated or moved from a first side of the substrate to a second side of the substrate by passing the polymer through a nanopore, pore or channel, e.g., by passing a single polymer through the nanopore, pore or channel. Polymers may be translocated through a nanopore, pore or channel, one polymer at a time. The polymer may include one or more labeled monomers. As the polymer and/or labeled monomer is moved or translocated through the nanopore, pore or channel and/or after the labeled monomer exits the nanopore, pore or channel, energy is transferred from a label positioned on the nanopore, pore or channel to the monomer label, resulting in energy being emitted from the monomer label. The emitted energy may be detected using an optical detector. Optionally, the optical detector may be operated in time delay integration mode or another mode. The energy emission may be associated with a particular or specific monomer such that the monomer may be identified based on detection of the energy emission. The sequence of the polymer may be deduced or determined based on detecting energy emission from translocated monomers of a polymer and identifying each monomer that makes up the polymer.

Referring back to FIG. 2C, a polymer sequencing method utilizing a nanopore, pore or channel, for example, biological nanopores, and a FRET reaction is depicted. A donor label 36 may be suitably positioned or localized at the base of the nanopore 32 and acceptor labels 38, e.g., fluorophores, may be attached to individual monomers making up the polymer, e.g., nucleotides of a nucleic acid, DNA and/or RNA. The nucleic acid is passed through the nanopore, resulting in energy transfer from the donor label to the acceptor label when the acceptor comes into proximity with the donor label or gets near the donor label. Energy emission from the acceptor label as a result of this energy transfer may be detected by an optical or photon detector, e.g., a high speed optical or photon detector.

In certain variations, biological nanopores, such as, e.g., membrane lytic protein alpha-hemolysin, may be used and may allow for precise reproducibility of pore dimensions, while providing a cost effective process for creating pores. The natural affinity of membrane proteins to lipid bilayers allows for positioning or localization of such nanopores to a lipid bilayer substrate.

In certain variations, systems and methods for performing optical molecule detection or polymer sequencing, e.g., optical nucleic acid sequencing, are provided. As described herein, a donor chromophore may be attached to a nanopore without obstructing the actual nanopore opening. Prior to the sequencing reaction, the nucleic acid may be tagged or labeled with acceptor chromophores, e.g., fluorescent molecules, one for each of the four building blocks or nucleotides. By applying an electric field across the nanopore, the labeled nucleic acid is threaded or translocated through or across the nanopore. As soon as the nucleic acid or acceptor chromophores comes in close proximity to the donor chromophore, a FRET (Foester Resonance Energy Transfer) reaction may occur between the donor and acceptor chromophores, which results in a distinct spectral and temporal photon emission from the nucleic acid or acceptor chromophore. Detection of this photon emission may allow for the analysis, identification or sequencing of the underlying or labeled nucleotide and/or nucleotide sequence. FRET is a mechanism of energy transfer between two molecules that are capable of absorbing and emitting photons by photo-excitation. The donor and/or acceptor molecules or chromophores may include fluorophores, capable of fluorescent emission, or quenching molecules, capable of quenching an emitted photon. Quenching may be performed where a donor chromophore, initially in its quantum optic excited state, transfers energy to an acceptor chromophore (in proximity, e.g., less than about 10 nm away from the donor) through non-radiative dipole-dipole coupling. The acceptor chromophore may return to a ground state by fluorescent emission resulting in the quenching of the donor emission.

The emission of energy, e.g., light of a specific wavelength, by an acceptor label or chromophore may be detected with the appropriate optical equipment or detection system in order to identify the labeled monomer, e.g., a labeled nucleotide, which corresponds to or is associated with the detected distinct or specific wavelength of emitted light.

Various optical detectors may be utilized to detect the emission of energy from a labeled molecule, e.g., a labeled monomer. For example, various optical detectors may be utilized to detect the emission of photons from a labeled nucleotide moving through a stationary or moving nanopore. In certain variations, various optical detectors or photon detectors as described herein may be utilized for optical detection of molecules, e.g., to perform optical nanopore sequencing.

In certain variations, an optical detector may include a photon detector such as an avalanche photo diode (APD). An APD is a highly sensitive semiconductor electronic device that exploits the photoelectric effect to convert light to electricity. An APD is a photo detector that provides a built-in first stage of gain through avalanche multiplication. By applying a high reverse bias voltage, e.g., in the range of about 100 V to about 200 V in silicon, an APD may demonstrate an internal current gain effect of about 100 due to impact ionization or an avalanche effect. Certain silicon APDs may employ alternative doping and beveling techniques compared to traditional APDs, allowing for greater voltage to be applied, e.g., greater than 1500 V, before breakdown is reached, and a greater operating gain, for example, greater than 1000.

In certain variations, an optical detector may include a photon detector such as a Single-Photon Avalanche Diode (SPAD) (also known as a Geiger-mode APD or G-APD). An SPAD is a solid-state photo detector based on a reverse biased p-n junction in which a photo-generated carrier can trigger an avalanche current due to the impact ionization mechanism. SPADs are specifically designed to operate with a reverse bias voltage well above the breakdown voltage, compared to APDs which operate at a bias lesser than the breakdown voltage.

Both APDs and SPADs are reverse biased semiconductor p-n junctions. However, APDs are biased close to, but below the breakdown voltage of the semiconductor. The high electric field provides an internal multiplication gain on the order of a few hundreds since the avalanche process is not diverging as in SPADs. The resulting avalanche current intensity is linearly related to the optical signal intensity. In an APD, a single photon may produce tens or hundreds of electrons.

An SPAD operates with a bias voltage above the breakdown voltage. Because the device is operating in this unstable above-breakdown regime, a single photon (or a single dark current electron) can set off a significant avalanche of electrons. In a SPAD, a single photon triggers a current in the mA region (i.e., billions of billions of electrons per second) which can be easily “counted”.

In certain variations, confocal microscopy may be utilized to provide optical detection. Confocal microscopy is an optical imaging technique used to increase optical resolution and contrast of an image by using point illumination and a spatial pinhole to eliminate out-of-focus light in specimens that are thicker than the focal plane. In a conventional (i.e., wide-field) fluorescence microscope, the entire specimen is flooded evenly in light from a light source. All parts of the specimen in the optical path are excited at the same time and the resulting fluorescence is detected by the microscope's photodetector or camera including a large unfocused background part. In contrast, a confocal microscope uses point illumination and a pinhole in an optically conjugate plane in front of a detector to eliminate any out-of-focus signals. The name “confocal” stems from this configuration. Because only light produced by fluorescence close to the focal plane may be detected, the image optical resolution, particularly in the sample depth direction, is better than that obtained using wide-field microscopes.

In certain variations, an optical detector may include a photon detector such as a charge coupled device (CCD) camera or electron multiplied charge coupled device (emCCD) camera.

In one variation, a CCD camera (e.g., an emCCD) may be operated in Time Delayed Integration (TDI) mode and utilized to perform optical nanopore detection or sequencing of a molecule or polymer. A camera in TDI mode may be used to capture photons from a moving object. When operating in TDI mode, a CCD may capture an image of a moving object while transferring integrated signal charges synchronously with the object's movement. This increases the collected signal by a factor equivalent to the number of TDI stages or transfers, thereby improving the signal-to-noise ratio by the square root of the number of TDI elements or stages. The implementation of TDI may allow for continuous movement of an object, e.g., a molecule, past the optical detector in one axis, which may yield a significant increase in throughput (i.e., the sample area scanned per unit time) over traditional stop-and-start stage and camera acquisition schemes.

In certain variations, an optical detector operated in TDI mode may capture photons emitted by a labeled DNA molecule moving through a stationary nanopore as a result of a FRET reaction between the nucleic acid label and a nanopore label. The continuous read-out provided by an optical detector in TDI mode allows for fast and sensitive analysis of one or more or multiple nanopores with a single optical detector. Optionally, one or more or multiple optical detectors in TDI mode may be utilized.

One or more or an array of optical detectors, such as (CCD) cameras, may be used in imaging systems. CCD cameras may be utilized to capture images of an area where two-dimensional information is acquired simultaneously.

In certain variations, a method for acquiring images of a 2-dimensional area is provided. An optical detector (e.g., a CCD camera) operated in time delay integration (TDI) mode may be provided and an object (e.g., a polymer or nucleic acid) may be translated laterally with respect to the optical detector in a direction parallel to the charge line transfer direction. The translation of the object may be synchronized with the line transfer rate of the optical detector. In TDI mode, the optical detector may accumulate and read out charges simultaneously, outputting image information line by line in a continuous manner at a high line rate, e.g., up to hundreds of thousands lines per second. For example, the TDI optical detector or camera may detect a fluorescent signal and convert the signal into an electrical charge which is then further processed. This is in contrast to an optical detector operated in a conventional mode where the optical detector is typically allowed to be exposed to a scene for a certain period of time to accumulate charges and then the charges stored in the pixels are read out in a separate step. Optical detectors, such as CCD cameras, may optionally be able to switch between the conventional and TDI modes, e.g., through software control or other user control.

In one variation, a CCD optical detector may be utilized to perform optical nanopore molecule detection or sequencing, e.g., nanopore nucleic acid, DNA or RNA sequencing. An array of nanopores may be utilized. For example, the nanopores may be arranged in a linear array. As polymer strands pass through the nanopores, the optical signals (e.g., photon emissions) from the polymers (e.g., from each labeled monomer) may be captured or detected by the CCD optical detector in parallel at high speed. This may be performed using a single CCD or emCCD optical detector, e.g., operated in TDI mode, where the optical detector can detect photon emissions at each nanopore simultaneously and differentiate between each photon emission.

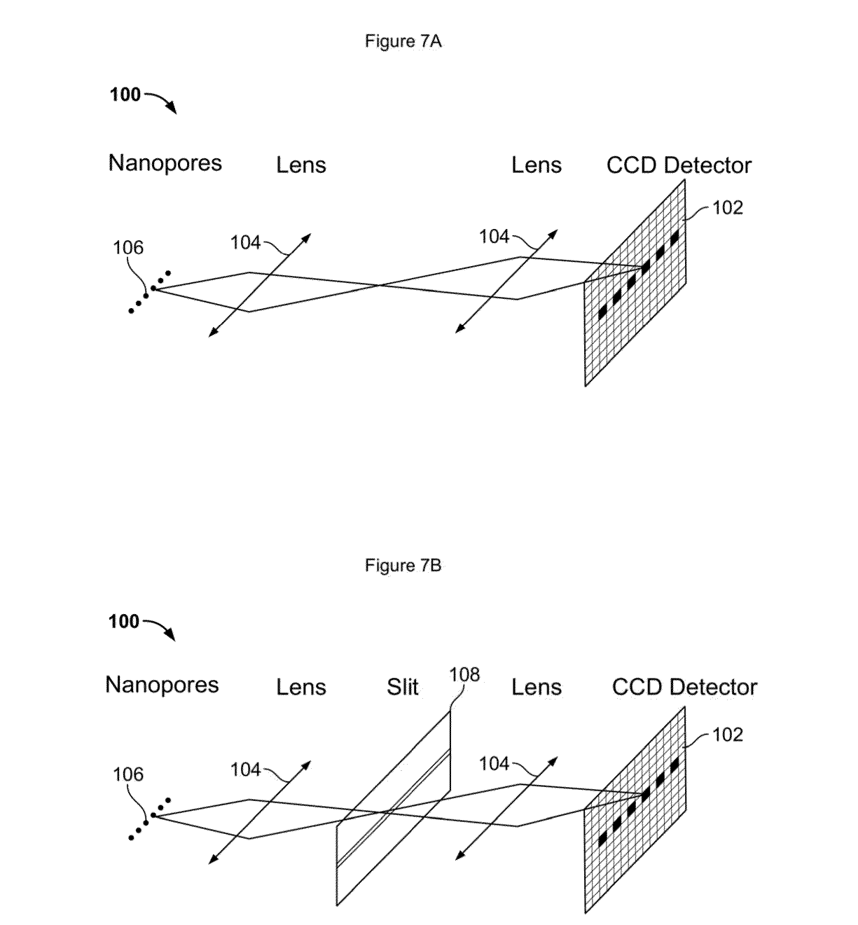

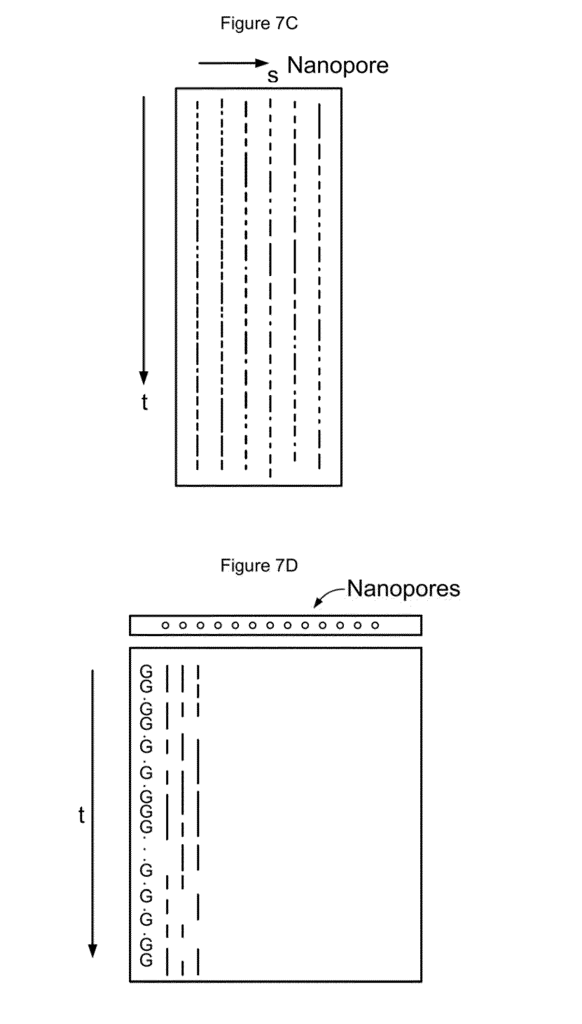

FIGS. 7A and 7B show a variation of a system 100 for performing optical nanopore molecule or polymer detection or sequencing. The system 100 may include one or more optical detectors 102, one or more lenses 104, one or more nanopores 106 and/or one or more field stops 108.

FIG. 7A shows an array of CCD detectors 102 which may or may not be operated in TDI mode. Lenses 104 may be positioned between the array of nanopores 106 and the CCD detectors 102. Before the initiation of molecule or polymer translocation through the nanopores, an image of the nanopore array 106 may be acquired to positively identify the location of the nanopores 106 and to ensure the alignment of the nanopore array 106 with the optical system, e.g., the CCD detectors 102 and/or lenses 104.

After the initiation of molecule or polymer strand translocation through the nanopores 106, a field stop 108 (as shown in FIG. 7B) may be inserted into the optical path to limit the view of the CCD detectors 102 and thereby reduce the detection of photons from outside the region of interest. A field stop 108 may reduce any background from photons generated from outside the region of interest, e.g., outside those photons emitted from a labeled monomer of the polymer as a result of a FRET reaction between a nanopore donor label and monomer acceptor label. The CCD detectors may be switched into TDI mode and the detected emission signals and corresponding charges are read out continuously line by line. Optionally, field stops may be inserted or positioned into an optical path before, after or during the initiation of polymer strand translocation. Optionally, the CCD or other optical detectors may be switched into TDI mode before, after or during initiation of polymer translocation.

The nanopores or pores may be kept stationary with respect to the optical system or optical detectors. The temporal information of the line scenes or the linear arrays of nanopores, may be captured or detected (as shown in FIGS. 7C and 7D). FIGS. 7C & 7D show charges corresponding to detected emission signals over time from translocated molecules, e.g., labeled nucleic acids, being red out continuously line by line as a result of optical detection by a detector, e.g., a CCD camera, operated in TDI mode. In FIGS. 7C & 7D, the nanopores through which the molecules are translated are shown along the X axis and time is shown along the Y axis. The detected temporal information of the photon emissions from each translocated labeled monomer, e.g., each labeled nucleotide, resulting from FRET reactions, allows for the analysis and/or sequencing of the translocating molecule or polymer (e.g., nucleic acid) made up of labeled monomers or a single monomer unit.

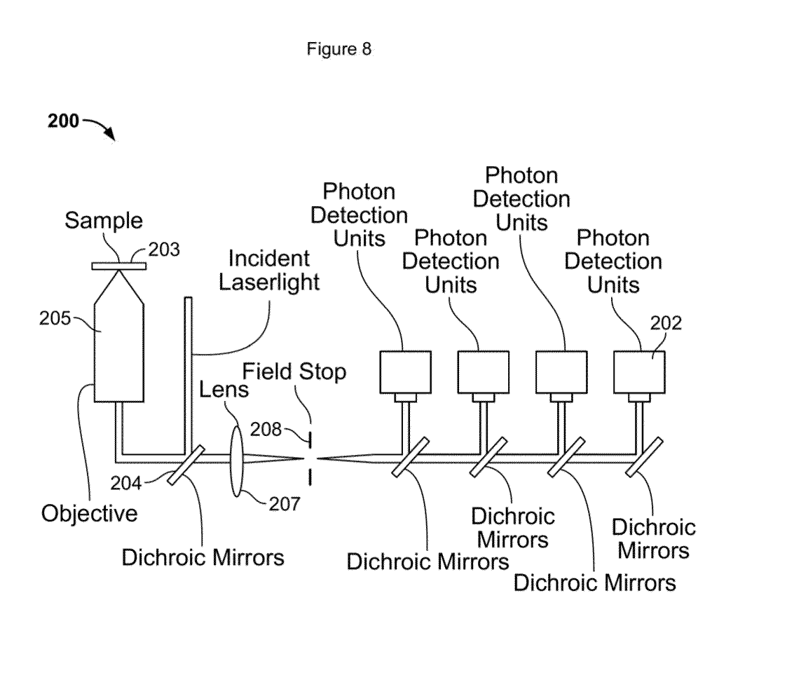

FIG. 8 shows another variation of a system 200 for performing optical single molecule or polymer detection through a nanopore or other opening, e.g. for performing optical polymer sequencing. The system 200 may allow for FRET light emission detection using highly sensitive photon detectors 202. The photon detectors 202 may or may not be operated in TDI mode. The system 200 may include a sample, for example, a molecule or polymer through one or more nanopores 203, an objective 205, e.g., a mechanism or microscope lens to guide light to the photon detectors or to collect photons, a lens 207 and one more mirrors 204, e.g., a dichroic mirror. A mirror 204 may be used to split emission light from the donor and the acceptor label, chromophore, or fluorophore into wavelengths for a donor signal and an acceptor signal, e.g., one or more of the four distinct acceptor signals for the four nucleotide bases of a nucleic acid. An incident laser light provides light through the objective to the nanopore to excite a donor label on the nanopore. A field stop 208 may be provided to reduce background light and to allow only light from the sample (nanopore or translocated molecule) to the photon detector. In one variation, where an APD is used as a photon detector, the small size of the photon detector surface may be used as a built in pinhole at the confocal plane to reject off-focal illumination. In another variation, where a CCD is used as a photon detector, a field stop 208 may be built into the optical path to eliminate stray light from outside the region of interest.

Various systems and/or methods for performing optical detection of molecules, e.g., optical sequencing of polymers, e.g., nucleic acids, utilizing pores or nanopores are described herein. In certain variations, optical systems or designs are provided which allow for the sequencing of one or more nucleic acids using a FRET reaction in combination with one or more nanopores. An optical detection system may detect energy or photons emitted from one or more nucleic acids translocated through a single or a multitude or array of nanopores for the purpose of sequencing one or more nucleic acid molecules.

In one variation, a system or method for optical detection or sequencing of a molecule may include one or more nanopores that allow for optical polymer or nucleic acid sequencing. The system may include one or more mirrors, e.g., dichroic mirrors. The mirrors may be used to split photons having various wavelengths between multiple photon detectors, where each wavelength may be associated with a specific or particular monomer and may be emitted from the labeled monomers of one more polymers translocated through a nanopore.

In another variation, a system or method for optical detection or sequencing of a polymer or other molecule may utilize an array of nanopores. The nanopores may be arranged as a parallel or linear array of nanopores. The detection of photons emitted from translocated polymers may be accomplished by one or more or an array of individually addressable photon detectors. Optionally, a photon detector may be positioned a distance from a nanopore such that one or more optical elements may be positioned in the path between the nanopore and the photon detector.

Systems or methods for optical detection or sequencing of a molecule, e.g., a polymer or nucleic acid, may utilize various optical or photon detectors. Examples of optical or photon detectors that may be utilized in the various systems or methods described herein may include but are not limited to one or more large area high speed Silicon Photo Avalanche Diodes; electron multiplied charge coupled device (emCCD) cameras; avalanche photo diodes (APD); photo or photon multipler tubes (PMT), a complementary metal oxide semiconductor (CMOS) detector, or a time delayed integration (TDI) camera. In certain variations, the emCCD camera may be operated in TDI mode. Any of the above detectors may be used to detect energy or photon emissions from one or more nanopore translocations of molecules, e.g., a parallel array of nanopore translocations.

In certain variations, photon emission from an acceptor label or chromophore of a monomer, such as a nucleotide, may be detected or captured by any of the optical or photon detectors or detection units described herein to perform optical nanopore sequencing.

A electric field may be applied across a nanopore, which may provide for a high translocation rate of nucleic acids through or across the nanopore. For example, unmodified single stranded DNA may move or translocate through an alpha hemolysin nanopore at approximately 200,000-500,000 nucleotides/sec. Given a maximum frame rate of standard commercial emCCD cameras of about 3000 fps (frames per second), such a device would not be able to read the unmodified nucleic acid sequence at single nucleotide resolution.

In certain variations, an optical detection system utilizing an optical detector, e.g., operated in TDI mode, may be utilized to perform optical nanopore detection of a labeled molecule, e.g., optical nanopore sequencing of a labeled or modified polymer, e.g., a labeled nucleic acid. For instance, an optical detector in TDI mode may scan up to about 200,000 fps, e.g., at about 10,000 to about 200,000 fps or at about 50,000 fps (frames per second). Optionally, the TDI optical detector may scan at a rate higher than 200,000 fps. The frame rate of a camera operated in TDI mode would be the line rate divided by the number of lines in that frame.

In certain variations, an optical detection system utilizing an optical detector may be utilized to perform optical nanopore detection of a labeled molecule, e.g., optical nanopore sequencing of a labeled or modified polymer, e.g., a labeled nucleic acid. For instance, an optical detector may scan up to about 200,000 fps, e.g., at about 2,000 to about 200,000 fps or about 2,000 to about 50,000 fps (frames per second). Optionally, the optical detector may scan at a rate higher than 200,000 fps. For example, a CMOS detector may scan from about 2,000 fps to about 50,000 fps or higher than 50,000 fps. Other optical detectors may be utilized as well.

A labeled or modified molecule or nucleic acid or DNA may translocate through a nanopore at about 500 to about 8000 nucleotides/sec or 1000 to about 5000 nucleotides/sec. An optical detector may detect the labeled nucleotide emissions at single nucleotide resolution and/or read the translocated labeled nucleotides or nucleic acid up to about 10× oversampling. An optical detector, e.g., an emCCD camera operated in TDI mode, may detect the labeled nucleotide emissions at single nucleotide resolution and/or read the translocated labeled nucleotides or nucleic acid at about 10× oversampling. An emCCD (electron-multiplying charge coupled device) camera or other optical detector or similar camera is able to detect energy or photon emissions from a plurality of nanopores simultaneously while differentiating between the plurality of energy emissions and detecting from which nanopore each emission comes from. For example, an emCCD camera has an integrated chip having multiple light sensitive pixels. The emCCD camera is able to take an image of the plurality of nanopores, and the nanopores, which may be labeled for identification purposes, will appear on the image, providing the geographic position or localization of the nanopores relative to the camera. Certain pixels of the emCCD chip will only collect light from certain nanopores based on the nanopore's position relative to the camera. Where energy emissions or photon bursts appear on the image will indicate from which nanopore the emissions are coming. An emCCD may differentiate or detect from which nanopore the energy is emitted based on the position or geographic location of the nanopore and energy emission relative to the camera or relative to or on the integrated chip of the camera. The emCCD has light sensitive pixels which may detect various wavelengths of light or energy and for differentiating between the various wavelengths. Furthermore, when the emCCD camera is operated in time delay integration mode, the frame rate or line rate of the camera is increased compared to the frame rate of the camera in non TDI mode.

In certain variations, a method of controlling or reducing the translocation speed of a molecule or polymer, e.g., a nucleic acid, through a nanopore, pore, opening or channel to increase the accuracy of and/or to allow for the detection, identification or sequencing of the translocated molecule is provided. For example, the translocation speed of a molecule or polymer through a nanopore or pore may be reduced or slowed by increasing the diameter of a molecule or polymer, e.g., by modifying one or more monomers of the polymer, e.g., by adding a label, tag, moiety or bulky group to the polymer. For example, a molecule or polymer, e.g., a nucleic acid, may be modified or labeled to reduce translocation speed of the molecule or polymer through a nanopore or pore in accordance with any of the variations of labeled or modified molecules or polymers described herein, e.g., such as the labeled molecules or nucleic acids shown and described in FIGS. 2A-5C. In certain variations, the increased diameter of the molecule may reduce the translocation speed of the molecule through a nanopore, pore, opening or channel sufficient to allow for single molecule analysis. In certain variations, reducing or slowing the translocation speed of a polymer through a nanopore may allow for or improve the optical detection of energy or photon emissions and/or allow for optical detection that differentiates between various energy or photon emissions from a translocated labeled molecule or monomer of a polymer to provide an optical read out to detect or sequence the translocated polymer. In certain variations, reducing or slowing the translocation speed of a polymer through a nanopore may allow for or improve electrical detection of current changes or drops through or in a nanopore and/or allow for electrical detection that differentiates between various current changes or drops through or in a nanopore to provide an electrical read out to sequence the translocated polymer, wherein the current changes or drops are associated with a particular monomer. Reducing the translocation speed of a molecule through a nanopore may be beneficial in performing optical and/or electrical molecule or polymer detection or sequencing either separately or in combination. In any of the above variations, the nanopores may or may not be electrically separated from one another.

The diameter of the molecule or polymer, e.g., a nucleic acid, may be increased by modifying one or more monomers of the polymer, e.g., by adding a label, tag, moiety or bulky group to the polymer. The modified polymer may then be translocated through the nanopore (where a single polymer may be translocated through the nanopore at a time) and the increased diameter of the polymer effects the interaction between the polymer and nanopore, for example, by causing the polymer to interact with or contact the nanopore as the polymer is threaded or translocated through the nanopore. This may reduce the speed at which the polymer moves or translocates through the nanopore. In certain variations, the polymer may be modified to increase the diameter of the polymer to reduce its translocation speed through a nanopore or other pore, and the nanopore may or may not be modified or otherwise altered to decrease the diameter of the nanopore passage or obstruct the passage in any way to affect polymer translocation speed.

In one variation, fluorescently or chromophore labeled nucleotides may be incorporated into a nucleic acid strand which results in an increased diameter of the nucleic acid. The measured translocation rate or speed of a single modified nucleic acid strand may be reduced compared to an unmodified nucleic acid, where the translocation speed of a modified nucleic acid may average about 1,000 to about 8,000 nucleotides/sec. A labeled or modified nucleic acid may reduce the translocation speed to a rate of about 10,000 units or nucleotides per second or less. For other modified molecules, translocation speed may be about 10,000 units per second or less. This reduction in translocation rate or speed makes optical read-out or detection of energy emissions from translocated nucleotides making up the nucleic acid at single nucleotide resolution via emCCD, APD, PMT or other photon detector based systems feasible and more accurate. Optical read-out or detection may be performed on multiple molecules or polymers being translocated simultaneously through an array of nanopores, e.g., a parallel array. Highly sensitive photon detectors, e.g., APDs or PMTs have a high acquisition rate and may be implemented in various systems, e.g., in a parallel acquisition set-up. APDs may have count speeds (=photons detected/sec) of >20 million/sec and PMTs may have similar rates. For example, a plurality or array of APDs or PMTs may be utilized to detect signals from a plurality or an array of nanopores simultaneously. Alternatively, a single emCCD camera operated in TDI mode (or non TDI mode if a molecule translocates through the nanopore at slow enough rate) may be utilized to detect signals from one or more or a multitude of nanopores simultaneously.

Various labels may be utilized to reduce the translocation speed of a polymer, e.g., a nucleic acid, through a pore or nanopore. Examples include but are not limited to: Cyanine™ dyes (e.g. Cy5, Cy3, Cy5.5, Cy7) Alexa™ Fluor labels, Atto™ dyes, and various fluorescent dyes which are derivatives of Xanthenes, Cyanines, Naphtalenes or Coumarines. Various labels may have various lengths, e.g., ranging from about 5 to about 20 Angstrom (0.5-2 nm). Labels may be attached directly or indirectly at various locations on a monomer or polymer. For example, labels may be bound to the base of a nucleic acid. A label may be attached or bound such that it may freely rotate or move relative to the monomer or polymer to which it is attached. For example, a label may rotate or flip back towards a DNA backbone when the labeled, single or double stranded DNA enters a nanopore. For example, the diameter of a labeled nucleic acid as it is being translocated through a nanopore may range from about 1.0 to about 5.0 nm or from about 1.2 to about 2.4 nm.

In certain variations, as described supra, a one or more or an array of nanopores, channels, pores or openings may be utilized in performing optical nanopore detection or sequencing of one or more molecules. For example, a method for sequencing a plurality of molecules or polymers may include one or more of the following steps: providing an one or more or an array of nanopores; translocating a plurality of molecules or polymers through the nanopores (e.g., where a single molecule passes through each nanopore simultaneously or sequentially); optically detecting a separate energy emission from each molecule or polymer at each nanopore simultaneously or sequentially, where each energy emission has a wavelength associated with a specific molecule or monomer; and/or identifying or sequencing the molecules or polymers based on the detected energy emissions. The optical detection may include differentiating or distinguishing between each emission of energy or each signal and detecting from which nanopore the energy is being emitted. One or more or all of the nanopores may or may not be electrically separated from each other.

In certain variations, a system or biosensor for sequencing or detecting a one or more or a plurality of molecules, e.g., a plurality of polymers, may include one or more or an array of nanopores, pores, channels or openings arranged on a substrate. An optical detector may be provided for detecting a separate energy emission from each molecule or polymer at each nanopore simultaneously or sequentially. For example, a single molecule may pass through each nanopore simultaneously or sequentially. Each energy emission may have a distinct wavelength associated with a specific molecule or monomer such that the molecules or polymers may be sequenced based on the detected energy emissions. The optical detector may differentiate or distinguish between each emission of energy or each signal and detect from which nanopore the energy is being emitted. One or more or all of the nanopores may or may not be electrically separated from each other.

In certain variations, a substrate may be provided with one or more or an array of nanopores or pores. Various arrangements or numbers of nanopores may be utilized. For example, a substrate may have 5 or more nanopores. A substrate may have a plurality or array of nanopores, where two or more or all of the nanopores are not electrically separated from one another. Optionally, an array or linear array of nanopores, e.g., 5 or more nanopores, may be provided on a substrate where the nanopores are not electrically separated from one another. Alternatively, a plurality or array of nanopores may be provided on a substrate where one or more of the nanopores are electrically separated from one another.

In certain variations, the nanopores may include a chromophore or fluorescent label. The labeled nanopores may undergo a FRET reaction with a labeled monomer of a polymer translocated through the nanopore. The labeled nanopores may be utilized for nanopore energy transfer polymer sequencing.

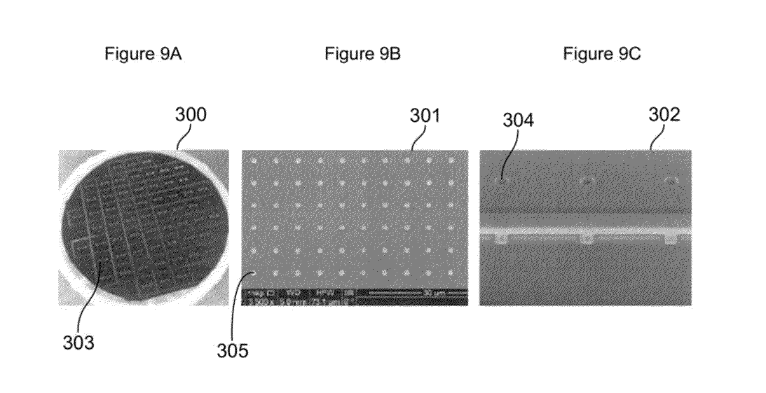

FIGS. 9A-9C show examples of substrates 300, 301 and 302 having arrays of nanopores 303, 304, 305 respectively, arranged in various configurations, e.g., arranged in various linear configurations. In certain examples, a plurality of nanopores or nanopore array may be positioned on a wafer or SEM substrate or chip. Each nanopore may have a diameter ranging from about 2 to about 20 nanometers or about 10 nanometers. Arrays of nanopores may also be generated by creating larger nanoholes, 500 nanometers to 200 micrometers or about 50 micrometer which are spanned by a lipid bilayer into which the nanopore protein may be inserted, thereby generating a 1-2 nanometer wide nanopore. Any of the substrates described in FIGS. 9A-9C or similar substrates my be utilized to perform molecule or polymer or nucleic acid detection or sequencing according to detection or sequencing methods described herein.

The various nanopore substrates described herein may be utilized for optical molecule or polymer detection or sequencing via translocation of molecules or polymers therethrough. In certain variations, a nanopore substrate may be utilized for performing nanopore energy transfer sequencing. The nanopore substrate may be utilized to perform single molecule optical sequencing. For example, a nanopore array may be utilized to sequence a nucleic acid or DNA at about 5,000 nucleotides per second. Performing nanopore energy transfer sequencing using an array of nanopores allows for ultra-high throughput, e.g., about 1000 nanopores may read about 3×10̂9 nucleotides in about 10 minutes. No external sample preparation may be required and a user may add whole genomic DNA to a sequencing cartridge or nanopore array where the labeling process starts prior to the sequencing reaction. Since no polymerases may be part of the actual sequencing reaction, long read lengths may be obtained, e.g., read lengths greater than 1 kb. Nanopore energy transfer sequencing provides low cost and high throughput polymer sequencing.

Any of the optical or electrical detection methods or systems described herein may allow for data acquisition or the acquisition of data regarding the identity, quantity, presence, concentration, abundance or other parameter or property of the detected molecule, e.g., a polymer such as nucleic acid. For example, optical detection may detect a photon of a specific wavelength that is associated with a specific nucleotide, where the detected photon is converted into a signal or charge used to identify or read the specific detected nucleotide or other molecule, e.g., utilizing software or hardware, to sequence or detect a nucleic acid or other molecule. In certain variations, optical detection of a molecule may allow for the continuous detection of energy emission from a translocated labeled molecule. The molecules may be detected while continuously moving in a single file or along a single axis through a nanopore, pore or channel and past the optical detector. For example, molecules or polymers such as nucleic acids may be detected or read or sequenced while continuously moving in a single file or along a single axis through a nanopore, pore or channel past an optical detector or other detector, e.g., past an optical detector operated in TDI mode.

In certain variations, a method of optically detecting a molecule may include one or more of the following steps: providing one or more nanopores on a substrate; translocating a labeled molecule through the nanopore; detecting an energy emission from the labeled molecule using an optical detector operated in time delay integration mode, where the energy emission is associated with a specific molecule; and deducing the identity of the molecule based on detection of the energy emission.

The optical detector may be a photon detector. The photon detector may be an electron multiplied charge coupled device (emCCD) or other optical detector. The optical detector may be operated in time delay integration mode which provides a continuous detection and readout of energy emissions. Energy emission may be detected from a labeled molecule after the labeled molecule passes through and exits the nanopore. Energy emission may be detected from a labeled molecule as the molecule is moving and the nanopore remains stationary, and a continuous detection or readout may be provided. Optical detection of a molecule may include exciting a donor label attached to the nanopore; and transferring energy from the excited donor label to an acceptor label on the molecule after the labeled molecule passes through and exits the nanopore, wherein the acceptor label emits energy to be detected. The donor label and acceptor label may undergo a FRET (Förster Resonance Energy Transfer) reaction.

A plurality of molecules may be translocated through a plurality or an array of nanopores simultaneously or sequentially (e.g., where a single molecule or more than one molecule may pass through each nanopore simultaneously or sequentially) and separate energy emissions from each molecule are detected simultaneously or sequentially. The nanopores may be arranged in one or multiple linear arrays. The energy emissions from the plurality of molecules may be detected by a single optical detector, wherein the optical detector can distinguish between each detected energy emission at each nanopore. The energy emissions from the plurality of molecules may be detected by a plurality or an array of optical detectors, each assigned to a specific nanopore. The energy emission may be a photon.

In certain variations, the labeled molecule may be a polymer, wherein the energy emission is detected from a labeled monomer of the polymer the energy emission being associated with a specific monomer, and wherein the identity of the polymer sequence may be deduced based on detection of energy emissions and identification of monomers making up the polymer. For example, the polymer may be a nucleic acid having a plurality of labeled nucleotides, wherein a photon emitted from each labeled nucleotide may be detected such that the optical detector can read the nucleic acid at single nucleotide resolution to sequence the nucleic acid. An energy emission may be detected from a labeled monomer of the polymer after the labeled monomer passes through and exits the nanopore. As the polymer is moving and the nanopore remains stationary. The method may include exciting a donor label attached to the nanopore; and transferring energy from the excited donor label to an acceptor label on the monomer after the labeled monomer passes through and exits the nanopore, wherein the acceptor label emits energy to be detected. The donor label and acceptor label undergo a FRET (Förster Resonance Energy Transfer) reaction.

A label may modify the molecule by increasing the diameter of the molecule, thereby reducing a translocation speed of the molecule through the nanopore to facilitate optical detection of the molecule. Reducing the translocation speed of the molecule through the nanopore may facilitate the optical detection of a plurality of molecules translocating through a plurality of nanopores simultaneously.

In certain variations, a method of optically detecting a plurality of molecules may include one or more of the following steps: providing one or more or a plurality or an array of nanopores; translocating a plurality of molecules through the nanopores; optically detecting a separate energy emission from each molecule at each nanopore simultaneously, wherein each energy emission has a wavelength associated with a specific molecule; and identifying the molecules based on the detected energy emissions.

The optical detection may include differentiating between each energy emission and detecting from which nanopore the energy is emitted. The nanopores are arranged in a linear configuration. The nanopores may or may not be electrically separated from one another. The optical detection of the plurality of molecules at the plurality of nanopores may be performed by a single optical detector. The optical detector may be operated in time delay integration mode. Optionally, the optical detection of the plurality of molecules at the plurality of nanopores may be performed by a plurality of optical detectors, each assigned to a specific nanopore. The molecule may be a polymer, wherein the energy emission is detected from a labeled monomer of the polymer the energy emission being associated with a specific monomer, and wherein the identity of a polymer sequence may be deduced based on detection of energy emissions and identification of monomers making up the polymer. In one example, the polymer may be a nucleic acid comprising a plurality of labeled nucleotides, wherein a photon emitted from each labeled nucleotide may be detected such that the optical detector can read the nucleic acid at single nucleotide resolution to sequence the nucleic acid.

In certain variations, a system for optically detecting a one or more or a plurality of molecules may include one or more or a plurality or an array of nanopores arranged on a substrate; and one or more optical detectors. The optical detectors may be configured to detect a separate energy emission from each molecule at each nanopore simultaneously, wherein each energy emission has a wavelength associated with a specific molecule.

The nanopores may be arranged in linear configuration. For example, the nanopores may be arranged in a parallel linear configuration. A single optical detector may be utilized, wherein the optical detector is configured to differentiate between each energy emission and detect from which nanopore the energy is emitted. The nanopores may or may not be electrically separated from one another. The optical detector may be operated in time delay integration mode.

In certain variations, a method of reducing the translocation speed of a molecule through a nanopore to allow for detection of the molecule may include one or more of the following steps: increasing the diameter of the molecule by modifying the molecule; and translocating a single molecule through the nanopore where the increased diameter of the molecule causes the molecule to interact with the nanopore, thereby reducing the speed at which the molecule translocates through the nanopore.

Modifying the molecule may include adding a label, tag, moiety or bulky group or other group to the molecule. The method may further include optically detecting energy or photon emissions from a translocated modified molecule, wherein the energy emission is associated with a specific molecule. The optical detection may be performed by various photon detectors, e.g., a silicon photo avalanche diode; an electron multiplied charge coupled device (emCCD); an avalanche photo diode (ADP); a photo or photon multipler tube (PMT), a complementary metal oxide semiconductor (CMOS) detector, or a time delayed integration (TDI) camera, or other optical detectors. The optical detection may optionally be performed using an optical detector operated in time delayed integration mode.

A plurality of labeled molecules may be translocated through a plurality or an array of nanopores simultaneously and separate energy emissions from each molecule may be detected simultaneously. The energy emissions from the plurality of molecules may be detected by a single optical detector, wherein the optical detector can differentiate between each detected energy emission. The energy emissions from the plurality of molecules may optionally be detected by a plurality or an array of optical detectors, each assigned to a specific nanopore. The method may optionally include detecting current changes or drops through or in a nanopore, wherein the current changes or drops are associated with a specific molecule.

The modified molecule may be a polymer, wherein the energy emission is detected from a labeled monomer of the polymer the energy emission being associated with a specific monomer, and wherein the identity of the polymer sequence may be deduced based on detection of energy emissions and identification of monomers making up the polymer. For example, the polymer may be a nucleic acid having a plurality of labeled nucleotides, wherein a photon emitted from each labeled nucleotide may be detected such that the optical detector can read the nucleic acid at single nucleotide resolution to sequence the nucleic acid.

In certain variations of methods and systems described herein where optical detection is utilized to detect molecules, such methods and systems may utilize solely optical read-outs or optical detection of energy emission or light emission by a labeled molecule or polymer or monomer to detect or identify the molecule or polymer and/or to sequence a molecule or polymer including monomers, e.g., a nucleic acid. In certain variations, optical read-outs or optical detection of molecules or polymers, e.g., nucleic acid, may be performed without the use of electrical or current readouts or detection.

In other variations, a combination of optical and electrical or current change readouts or detection may be utilized to detect or identify molecules.

Each of the individual variations described and illustrated herein has discrete components and features which may be readily separated from or combined with the features of any of the other variations. Modifications may be made to adapt a particular situation, material, composition of matter, process, process act(s) or step(s) to the objective(s), spirit or scope of the present invention.

Methods recited herein may be carried out in any order of the recited events which is logically possible, as well as the recited order of events. Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

This disclosure is not intended to be limited to the scope of the particular forms set forth, but is intended to cover alternatives, modifications, combinations of elements disclosed in different variations, and equivalents of the variations described herein. Further, the scope of the disclosure fully encompasses other variations that may become obvious to those skilled in the art in view of this disclosure. The scope of the present invention is limited only by the appended claims.

1.-75. (canceled)

76. A method of optically detecting a molecule comprising:
providing one or more pores on a substrate;
translocating a labeled molecule through the pore;
detecting an energy emission from the labeled molecule using an optical detector, wherein the energy emission is associated with a specific molecule; and
deducing the identity of the molecule based on detection of the energy emission.

77. The method of claim 76, wherein the detector is a photon detector such as an electron multiplied charge coupled device (emCCD).

78. The method of claim 76, further comprising detecting an energy emission from a labeled molecule after the labeled molecule passes through and exits the pore.

79. The method of claim 76, further comprising detecting an energy emission from a labeled molecule as the molecule is moving and the pore remains stationary.

80. The method of claim 76, wherein the labeled molecule is a polymer, wherein the energy emission is detected from a labeled monomer of the polymer the energy emission being associated with a specific monomer, and wherein the identity of the polymer sequence is deduced based on detection of energy emissions and identification of monomers making up the polymer.

81. The method of claim 80, wherein the polymer is a nucleic acid comprising a plurality of labeled nucleotides, wherein a photon emitted from each labeled nucleotide is detected such that the optical detector can detect or read the nucleic acid at single nucleotide resolution to sequence the nucleic acid.

82. The method of claim 80, further comprising detecting an energy emission from a labeled monomer of the polymer after the labeled monomer passes through and exits the pore.

83. The method of claim 80, further comprising:
exciting a donor label attached to the pore; and
undergoing a FRET (Förster Resonance Energy Transfer) reaction from the excited donor label to an acceptor label on the monomer after the labeled monomer passes through and exits the pore, wherein the acceptor label emits energy to be detected.

84. The method of claim 76, wherein the label modifies the molecule by increasing the diameter of the molecule, which causes the molecule to interact with the nanopore thereby reducing a translocation speed of the molecule through the pore to facilitate optical detection of the molecule.

85. The method of claim 84, wherein reducing the translocation speed of the molecule through the pore facilitates the optical detection of a plurality of molecules translocating through a plurality of pores simultaneously.

86. The method of claim 76, wherein the pore is a nanopore, orifice or through hole in a substrate, the pore having a diameter of about 1 to about 10 nm which allows only the single file passage of molecules therethrough.

87. The method of claim 76, wherein solely optical detection is used to detect the molecule.

88. A method of optically sequencing a nucleic acid comprising:
providing a substrate having a plurality of nanopores;
translocating a plurality of labeled nucleic acids in single file through the plurality of nanopores simultaneously;
detecting separate energy emissions from each labeled nucleotide of a nucleic acid simultaneously after the labeled nucleotide passes through and exits the nanopore using an optical detector, wherein an energy emission is associated with a specific nucleotide and wherein the optical detector can differentiate between each detected energy emission and detect at which nanopore the energy is emitted; and
sequencing the nucleic acid based on detection of the energy emissions from the labeled nucleotides.

89. The method of claim 88, wherein solely optical detection is used to sequence the nucleic acid and the optical detector is an emCCD camera.

90. The method of claim 88, wherein the nanopores are not electrically separated from one another.

91. The method of claim 88, wherein an energy emission is detected from a labeled nucleotide as the nucleic acid is moving and the nanopore remains stationary.

92. The method of claim 88, further comprising:
exciting a donor label attached to the nanopore; and
transferring energy from the excited donor label to an acceptor label on the nucleotide after the labeled nucleotide passes through and exits the nanopore, wherein the acceptor label emits energy to be detected.

93. The method of claim 92, wherein the donor label and acceptor label undergo a FRET (Förster Resonance Energy Transfer) reaction.

94. The method of claim 88, wherein the nanopore is an orifice or through hole having a diameter of about 1 to about 10 nm which allows only the single file passage of nucleic acids therethrough.

95. The method of claim 88, wherein the optical detector may detect the labeled nucleotide emissions at single nucleotide resolution and at a rate that provides about 10× oversampling.


SEQUENCE LISTING (1 sequence)

SEQ ID NO: 1 - DNA, 17 bp, Artificial Sequence
  Sample nucleic acid sequence
gaagtttagttacagcc